(19) United States
(12) Patent Application Publication
(10) Pub. No.: US 2005/0283144 A1
(43) Pub. Date: Dec. 22, 2005

(54) Energy irradiating medical equipment, energy irradiating medical apparatus and irradiation method

(75) Inventors: Hiroshi Shiono, Tokyo (JP); Akira Sakaguchi, Kanagawa (JP); Yuichiro Irisawa, Kanagawa (JP)

Correspondence Address: BUCHANAN INGERSOLL PC;(INCLUDING BURNS, DOANE, SWECKER & MATHIS), POST OFFICE BOX 1404, ALEXANDRIA, VA, 22313-1404, US

(73) Assignee: Terumo Kabushiki Kaisha, Tokyo (JP)

(21) Appl. No.: 11/198,508

(22) Filed: Aug. 8, 2005

Related U.S. Application Data
(63) Continuation-in-part of application No. 11/090,241, filed on Mar. 28, 2005.

(30) Foreign Application Priority Data
Mar. 30, 2005 (JP) 2005-099305
Mar. 31, 2004 (JP) 2004-106089

(51) Int. Cl. A61B 18/18

(52) U.S. Cl. 606/18

(57) ABSTRACT

The present invention provides an energy irradiating medical equipment having an insert portion to be inserted into a living body, a temperature sensor disposed on the insert portion, and an energy irradiation window disposed on the insert portion for applying an energy to a living tissue. The temperature sensor includes a flexible thin-film substrate, at least first and second conductors disposed on the thin-film substrate, and a temperature measuring unit electrically coupled to the at least first and second conductors. The temperature measuring unit is disposed on the energy irradiation window.

This application is a continuation-in-part application of U.S. application Ser. No. 11/090,241 filed on Mar. 28, 2005, the entire content of which is incorporated herein by reference.

### FIELD OF THE INVENTION

The present invention generally relates to a medical apparatus for irradiating living tissue with energy to treat or diagnose the living tissue. More particularly, the invention relates to an energy irradiating medical apparatus and an energy irradiating medical equipment used thereof including a temperature sensor disposed in an insert portion to be inserted into a living body for accurately measuring the temperature of the living body, which it is being irradiated with an energy during treatment or diagnosis, without the need for thrusting into the living body. The invention also pertains to a method of irradiating living tissue in a living body.

### BACKGROUND DISCUSSION

There have been known in the art energy irradiating medical apparatus having an elongate insert portion to be inserted into a living body through a body cavity or a small incision. When the insert portion is inserted into the living body, the insert portion irradiates a living tissue including an affected region with an energy such as a laser beam, a microwave, a radio wave, an ultrasonic wave, or the like to thermally modify, necrose, coagulate, cauterize, or evaporate the tissue of the affected region or a surrounding tissue including the affected region.

The energy irradiating medical apparatus generally directly apply the energy to the surface layer of a living tissue or the affected region positioned closely thereto. The energy irradiating medical apparatus are also used to treat, with heat, an affected region positioned deeply in a living tissue, such as a prostatic hypertrophy, a prostatic cancer, or a prostatitis.

For example, International Application Publication No. WO93/04727 discloses a technique proposing a process of applying a laser beam to solidify or contract some tissue of a cancer or a prostate. According to this technique, a coolant is introduced into a balloon to prevent the surface of a urethra held in contact with the balloon from being heated, while only the cancer or the prostate located inside is being heated. However, since the laser beam is applied from a fixed laser beam irradiator, the laser beam needs to be applied at a low output level to prevent the surface of a urethra from being heated. Hence, the laser beam needs to be applied for a long period of time. International Publication No. WO93/04727 reveals a balloon catheter having a thermocouple disposed in the balloon to be located in an intermediate position in a prostatic urethra for monitoring the temperature of a urethral tissue. The thermocouple is disposed within the balloon and held out of direct contact with the urethra, and the coolant is circulated through the balloon. Therefore, the temperature measured by the thermocouple does not appear to be accurately representative of the temperature of the prostatic urethra. U.S. Pat. No. 5,964,791 discloses a process of thrusting into a prostate with a temperature sensor to accurately measure the temperature of the urethra (direct measuring process).

US Patent No. U.S. Pat. No. 6,579,286 discloses, as an example of heat treatment device, a laser beam irradiating apparatus for guiding a laser beam into a urethra to treat a prostatic hypertrophy. The laser beam irradiating apparatus has a laser beam irradiation portion that is continuously movable to change the direction of the applied laser beam at all times. However, since the laser beam irradiating apparatus is arranged to concentrate the laser beam on a target region, the target region is heated to a high temperature while holding a surrounding tissue around the target region at a lower temperature. Even if the target region is positioned deeply in the living tissue, therefore, any damage to the living tissue that is located between the laser beam irradiator and the target region is minimized.

A therapeutic procedure for treating a prostatic hypertrophy with the laser beam irradiating apparatus will be described below. First, the doctor inserts the insert portion of the laser beam irradiating apparatus into the urethra of the patient. The insert houses therein a laser beam irradiator having a reflecting surface for reflecting a laser beam which is generated by a laser beam generator, guided by an optical fiber, and emitted from the tip end of the optical fiber. The insert portion also houses therein an endoscope, and inlet outlet pipes for a coolant for cooling the laser beam irradiator. Then, the doctor positions the laser beam irradiator while observing the urethra with the endoscope in the insert through an observation window disposed in the insert, and then applies the laser beam to a target region in the patient.

The heat treatment device referred to above needs to measure the temperature of a treated region in order to monitor the treatment in progress. The temperature of the treated region (the target region to be irradiated with the laser beam) positioned deeply in the living body can be measured by a process of thrusting into the living tissue with a temperature sensor to directly measure the temperature of the deep region (direct measuring process) or a process of bringing a temperature sensor into contact with the surface layer of the living body above the treated region to accurately measure the temperature of the surface layer of the living body and estimating the temperature of the deep region based on the measured temperature.

Though the direct measuring process is able to accurately measure the temperature of the treated region, it is disadvantageous in that it invites side effects such as hemorrhage and infectious disease because the living body is injured by being pierced with the temperature sensor, resulting in an increased number of days that the patient needs to stay in the hospital. For this reason, there has been a demand for a technique to accurately measure the temperature of the surface of the living body while it is being treated by an energy irradiation, thereby increasing the accuracy to estimate the temperature of a deep living tissue.

Problems that arise regarding the accurate measurement of the temperature of the surface of the living body will be described below. Conventional Temperature sensors have a temperature measuring element such as a thermistor and two leads connected thereto, which are placed in a tangle-free manner in a protective tube. However, the protective tube makes the insert portion to be inserted into the living body thick, posing an increased burden on the patient. The leads that are employed tend to cause the thermistor to be installed in different positions, making it impossible to measure accurate temperatures.

It may be proposed to place the temperature measuring element and the leads within the insert portion. If the temperature measuring element is placed in the insert portion of an energy treatment device where a coolant is circulated in the insert portion for cooling an energy emission unit and the living body contacted by the insert portion, then the coolant affects the temperature measuring element. Consequently, there has been desired a temperature sensor less susceptible to the coolant and is yet capable of accurately measuring the surface temperature of a living body.

One solution would be to attach the temperature measuring element and the leads to the outer surface of the insert. However, this approach needs to meet the following requirements:

1. The temperature measuring element will not be affected by the coolant.

2. The temperature measuring element will be installed easily and accurately in a desired position.

3. When the temperature measuring element and the leads are attached, the leads will not be damaged and will keep electrically connected to the temperature measuring element.

4. The insert portion will not have protrusions on its surface, which would otherwise be liable to damage the living body when the insert portion is inserted into the living body.

5. The temperature measuring element will not be directly affected by the energy that is applied to the living body.

### SUMMARY

It is therefore an object of the present invention to provide an energy irradiating medical apparatus and an energy irradiating medical equipment used thereof having a structure that is simple and inexpensive to manufacture and capable of accurately measuring the temperature of a living tissue when the doctor treats a prostatic hypertrophy or a prostatic cancer with heat, using the energy irradiating medical apparatus.

An energy irradiating medical equipment according to an embodiment of the present invention has an insert portion to be inserted into a living body, a temperature sensor disposed on the insert portion, and an energy irradiation window disposed on the insert portion for applying an energy to a living tissue. The temperature sensor includes a flexible thin-film substrate, at least first and second conductors disposed on the thin film substrate, and a temperature measuring unit electrically coupled to the at least first and second conductors. The temperature measuring unit is disposed on the energy irradiation window.

Preferably, the temperature measuring unit is disposed in a peripheral region within the energy irradiation window.

Preferably, the temperature measuring unit has first and second electrodes bonded and electrically coupled respectively to at least the first and second conductors disposed on the thin-film substrate, and a substantially plate-shaped thermistor element made of a metal oxide. The first and second electrodes are electrically coupled to the thermistor element.

Preferably, the thermistor element is made of an oxide of one of transition metals including Mn, Co, Ni, and Fe.

Preferably, the thermistor element has a first surface disposed on the first electrode, the first electrode is bonded and electrically coupled to the thermistor element, the thermistor element has a second surface opposite to the first surface, with the second electrode being disposed on the second surface, and the second electrode is not bonded to, but electrically coupled to the thermistor element.

Preferably, the flexible thin-film substrate is bent to place the second electrode on a second surface of the thermistor element which is opposite to a first surface.

Preferably, the thin-film substrate is disposed outwardly of the energy irradiation window and along a longitudinal direction of the insert portion.

Preferably, the energy irradiating medical equipment further has an output tube covering the insert portion. After an outer surface of the insert portion is covered with the outer tube, the outer tube is thermally shrunk to press the thermistor element and the second electrode against each other to electrically couple the thermistor element and the second electrode to each other.

Preferably, the energy irradiating medical further has a thin metal film for shielding the thermistor element from the energy.

Preferably, the thin metal film is disposed on the thin-film substrate, and the thin-film substrate is bent to cover the thermistor element with the thin metal film.

Preferably, the insert portion has a hollow cylinder and an opening portion defined in a side wall of the hollow cylinder to provide the energy irradiation window.

Preferably, an optically transparent resin film is applied to the hollow cylinder in covering relation to the opening portion.

Preferably, the resin film is scaled.

Preferably, the energy irradiating medical equipment further has an outer tube covering the resin film.

Preferably, the thin-film substrate has depth markers for indicating the length by which the insert portion is inserted into the living body by the user.

Preferably, the temperature measuring unit is disposed in a peripheral region within the energy irradiation window, and a plurality of the temperature sensors are disposed in different positions on the insert portion.

An energy irradiating medical equipment according to an embodiment of the present invention has an insert portion to be inserted into a living body, a temperature sensor disposed on the insert portion, and an energy irradiation window disposed on the insert portion for applying an energy to a living tissue. The temperature sensor includes a flexible thin-film substrate, at least first and second conductors disposed on the thin-film substrate, and a temperature measuring unit electrically coupled to the at least first and second conductors and including a thin-film metal resistor, the temperature measuring unit being disposed on the energy irradiation window.

Preferably, the temperature measuring unit is disposed in a range greater than the irradiated width of the energy which passes through the energy irradiation window.

Preferably, the thin-film metal resistor is made of one of metals including Al, Pt, Ti, W, Ni, Ag, Au, and Cu, or an alloy thereof.

Preferably, each of the first and second electrodes includes a thin metal film which is made of the same material as the thin-film metal resistor.

Preferably, the thin-film metal resistor and the first and second electrodes are made of Al.

Preferably, the thin-film metal resistor and the first and second electrodes are formed by deposition of Al on the thin-film substrate.

Preferably, the thin-film substrate is made of a optically transparent resin for passing the energy.

Preferably, the optically transparent resin is one of polyester, polycarbonate, and polyethylene terephthalate (PET).

Preferably, the thin-film metal resistor covers the energy irradiation window in a range greater than the diameter of an irradiated spot of the energy, and smaller than the width of the energy irradiation window.

Preferably, the thin-film metal resistor is in the form of a thin line having a width in the range from 10 to 20 μm and a length in the range from 50 to 100 mm.

Preferably, the thin line is made of Al and has a resistance in the range from 100 to 1000 Ω.

An energy irradiating medical apparatus according to the present invention has an insert portion to be inserted into a living body, a temperature sensor disposed on the insert portion, and an energy irradiation window disposed on the insert portion for applying an energy to a living tissue. The temperature sensor includes a flexible thin-film substrate, at least first and second conductors disposed on the thin-film substrate, and a temperature measuring unit electrically coupled to the at least first and second conductors. The temperature measuring unit is disposed on the energy irradiation window. The energy irradiating medical apparatus has maximum surface temperature estimating means for estimating a maximum surface temperature of a living tissue which is irradiated with the energy, based on a temperature measured by the temperature sensor.

Preferably, the temperature measuring unit is disposed in a peripheral region within the energy irradiation window, the temperature measuring unit having first and second electrodes bonded and electrically coupled respectively to at least the first and second conductors disposed on the thin-film substrate, and a substantially plate-shaped thermistor element made of a metal oxide, the first and second electrodes being electrically coupled to the thermistor element.

Preferably, the temperature measuring unit is disposed on the energy irradiation window, the temperature measuring unit having first and second electrodes disposed on the thin-film substrate and bonded and electrically coupled respectively to at least the first and second conductors, and a thin-film metal resistor bonded and electrically coupled to the first and second electrodes.

Preferably, the energy irradiating medical apparatus further has deep region temperature estimating means for estimating a deep region temperature of a living tissue which is irradiated with the energy, based on a temperature measured by the temperature sensor.

Preferably, the energy irradiating medical apparatus further has control means for controlling the energy applied to the living tissue based on the temperature measured by the temperature sensor.

Preferably, the energy irradiating medical apparatus further has irradiating means disposed in the insert portion for reflecting the laser beam with a reflecting surface and applying the laser beam through the energy irradiation window to the living tissue, moving means for reciprocally moving the irradiating means along a longitudinal direction of the insert portion, changing means for changing an irradiation angle of the irradiating means, and determination means for determining whether or not the reciprocating movement of the irradiating means is correctly controlled by the moving means, based on the temperature measured by the temperature sensor.

Preferably, the energy is a laser beam.

According to another aspect an energy irradiating medical equipment comprises an insert portion possessing a size permitting the insert portion to be inserted into a living body, with the insert portion comprising a hollow cylinder possessing an interior. An energy emitter is positioned in the interior of the hollow cylinder and is adapted to be connected to an energy generator to emit energy. A temperature sensor is disposed on the insert portion, an opening is provided in the hollow cylinder, and a cover covers the opening and permits transmission therethrough of the energy emitted by the energy emitter. The temperature sensor comprises a flexible thin-film substrate, at least first and second conductors disposed on the thin-film substrate, and a temperature measuring unit electrically coupled to the at least first and second conductors. The temperature measuring unit is positioned outside of the cover so that the cover is positioned between the interior of the insert portion and the temperature measuring unit

In accordance with another aspect, energy irradiating medical equipment comprises an insert portion possessing a size permitting the insert portion to be inserted into a living body, with the insert portion comprising a hollow cylinder having an interior. An energy emitter is positioned in the interior of the hollow cylinder and is adapted to be connected to an energy generator to emit energy to irradiate living tissue of the living body, and a temperature sensor is disposed on the insert portion. The temperature sensor comprises a flexible thin-film substrate, at least first and second conductors disposed on the thin-film substrate, with the at least first and second conductors being positioned exteriorly of the hollow cylinder, and a temperature measuring unit electrically coupled to the at least first and second conductors.

According to a further aspect, a method for irradiating living tissue of a living body involves inserting into the living body an insert portion in which a temperature sensor is disposed on the insert portion, with the temperature sensor comprising a flexible thin-film substrate, at least first and second conductors disposed on the thin-film substrate and a temperature measuring unit electrically coupled to the at least first and second conductors. The method also involves emitting energy from within the insert portion and through a window in the insert portion to irradiate the living tissue with the energy, and determining a temperature in the living body using output from the temperature sensor.

The energy irradiating medical apparatus, energy irradiating medical equipment and method according to the present invention permits accurate measurement of the temperature of a living tissue as it is treated with heat, though the energy irradiating medical apparatus through use of an apparatus and equipment that are relatively simple in structure and relatively inexpensive to manufacture. Therefore, the doctor who operates the energy irradiating medical apparatus can correctly monitor the temperature of the living tissue as it is treated with heat to cure a prostatic hypertrophy, for example, and hence can treat the living tissue with greater safety.

### BRIEF DESCRIPTION OF THE DRAWING FIGURES

These and other objects of the invention will be seen by reference to the description, taken in connection with the accompanying drawing, in which:

FIG. 1 is a view, partly in block form, of a system arrangement of an energy irradiating medical apparatus according to an embodiment of the present invention;

FIG. 2 is a cross-sectional view of an insert of the energy irradiating medical apparatus;

FIG. 3 is a perspective view showing an internal structure of the insert;

FIG. 4 is an elevational view of a temperature sensor disposed on a hollow cylinder;

FIG. 5 is a fragmentary exploded perspective view illustrative of a process of forming a laser beam irradiating window using a graduated glass strip and then placing a temperature sensor on a hollow cylinder;

FIG. 6 is a fragmentary exploded perspective view illustrative of a process of forming a laser beam irradiating window using a graduated window seal and then placing a temperature sensor on a hollow cylinder;

FIG. 7A is a front elevational view showing a structure of the temperature sensor;

FIG. 7B is a cross-sectional view taken along line A-A of FIG. 7A;

FIG. 7C is a transverse cross-sectional view showing the temperature sensor placed on the hollow cylinder;

FIGS. 8A through 8D are views illustrative of a process of manufacturing the temperature sensor;

FIG. 9 is a view illustrative of the relationship between the movement of a reflecting surface and a living tissue region (target point) where a laser beam is concentrated;

FIGS. 10A through 10C are transverse cross-sectional views showing the relationship between the positions of nonparallel grooves at different cross-sectional positions;

FIG. 11 is a block diagram of a control circuit of the energy irradiating medical apparatus;

FIG. 12 is a diagram showing the correlation between measured values of the surface temperature and measured values of the maximum temperature of the lumen when a laser beam is applied;

FIG. 13 is a diagram showing measured values of the surface temperature Tu when a laser beam is applied at desired times and estimated values, calculated according to an equation (1), of the maximum temperature of the lumen and measured values thereof;

FIG. 14 is a flowchart of a process of calculating the maximum temperature Tmax of the lumen from the surface temperature Tu when a laser beam is applied;

FIG. 15 is a diagram showing measured values of the surface temperature Tu when a laser beam is applied at desired times and estimated values, calculated according to an equation (2), of the temperature of a deep region in a living body and measured values thereof;

FIG. 16 is a flowchart of a process of calculating the temperature Tp of the deep region in the living body from the surface temperature Tu when a laser beam is applied;

FIG. 17 is a diagram showing temperature changes caused in 2 seconds when laser beams are applied at output levels of 4, 11, and 16 W;

FIG. 18 is a flowchart of a process of determining whether irradiation timing is correct or incorrect from the surface temperature Tu when a laser beam is applied;

FIG. 19 is a diagram showing a temperature rise pattern Tutarget(t) of the surface temperature when a laser beam is applied and measured values of the surface temperature Tu(t);

FIG. 20 is a flowchart of a process of controlling a laser beam output level from the surface temperature Tu when a laser beam is applied; and

FIG. 21 is an elevational view of three independent temperature sensors disposed on one thin-film substrate.

FIG. 22 is an elevational view of a temperature sensor (aluminum sensor) disposed on a hollow cylinder;

FIG. 23 is a fragmentary exploded perspective view illustrative of a process of forming a laser beam irradiating window using a glass strip with scale and then placing a temperature sensor (aluminum sensor) on a hollow cylinder;

FIG. 24A is a front elevational view showing a structure of the temperature sensor (aluminum sensor);

FIG. 24B is an enlarged fragmentary transverse cross-sectional view of the temperature sensor (aluminum sensor);

FIG. 24C is a cross-sectional view of the temperature sensor (aluminum sensor) mounted on the hollow cylinder;

FIG. 25 is a diagram showing temperature values measured using the temperature sensor (aluminum sensor);

FIG. 26 is a diagram showing temperature values measured using an aluminum sensor and a thin thermistor as the temperature sensor;

FIG. 27 is a diagram showing values plotted at desired times of the surface temperature Tu when a laser beam is applied and estimated values plotted of the urethra surface temperature;

FIG. 28 is a diagram illustrative of a process of estimating the urethra surface temperature from the surface temperature Tu; and

FIG. 29 is a flowchart of a processing sequence for calculating the urethra surface temperature Tmax from the surface temperature Tu when the laser beam is applied.

### DETAILED DESCRIPTION OF THE INVENTION

A first embodiment of the present invention will be described below as being applied to an energy irradiating medical apparatus for treating, with heat, a prostatic hypertrophy. However, the principles of the present invention are not limited to such an energy irradiating medical apparatus for treating, with heat, a prostatic hypertrophy. A laser beam will be described as an example of energy used for treating, with heat, a prostatic hypertrophy. However, the energy is not limited to a laser beam, but an electromagnetic wave such as a microwave, a radio wave, or the like, or an elastic wave such as an ultrasonic wave, a sound wave, or the like may be used as the energy.

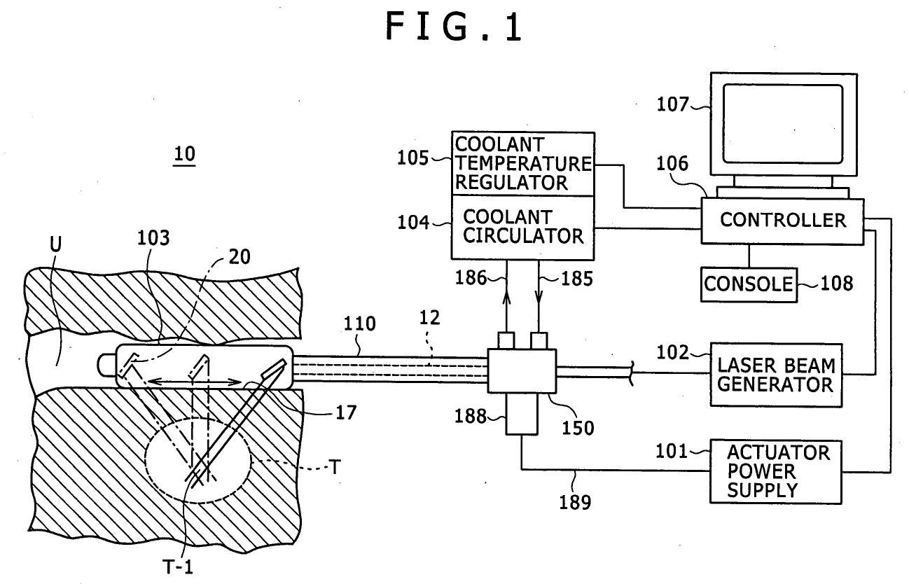

The laser beam that can be used may include a divergent beam, a parallel beam, or a convergent beam. An optical system for converting a laser beam into a convergent beam may be disposed in the path of the laser beam. Though the laser beam is not limited to any particular laser beams insofar as they can reach a deep region in a living body, the laser beam should preferably have a wavelength ranging from 500 to 2600 nm, more preferably from 750 to 1300 nm or from 1600 to 1800 nm. The laser beam may be generated by a gas laser such as an He—Ne laser or the like, a solid-state laser such as an Nd-YAG laser or the like, or a semiconductor laser such as a GaAlAs laser or the like.

[Energy Irradiating Medical apparatus (FIG. 1)]

FIG. 1 shows, partly in block form, of a system arrangement of an energy irradiating medical apparatus 10 according to an embodiment of the present invention.

As shown in FIG. 1; the energy irradiating medical apparatus 10 is a lateral-emission laser beam irradiating apparatus, and includes an applicator 110 having an insert portion 103 to be inserted into a body cavity U such as an urethra, for example. The insert portion 103 is mounted on the distal end of the applicator 110, and an outside diameter of the insert portion 103 is not limited to any values insofar as it can be inserted into the body cavity U. However, the outside diameter of the insert portion 103 should preferably be in the range from 2 to 20 mm, and more preferably in the range from 3 to 8 mm.

The insert portion 103 houses therein a laser beam irradiation portion 20 that is reciprocatingly movable in the longitudinal direction of the insert portion 103. A laser beam is guided by an optical fiber 12 extending through the applicator 110 and emitted from the distal end of the optical fiber 12. The laser beam emitted from the optical fiber 12 is reflected by the laser beam irradiation portion 20 and applied through a laser beam irradiating window defined in a side wall of the insert portion 103 to a target region T-1 to be irradiated in a living tissue T. The optical fiber forms an energy emitter from which energy (e.g., a laser beam in this disclosed embodiment) is emitted.

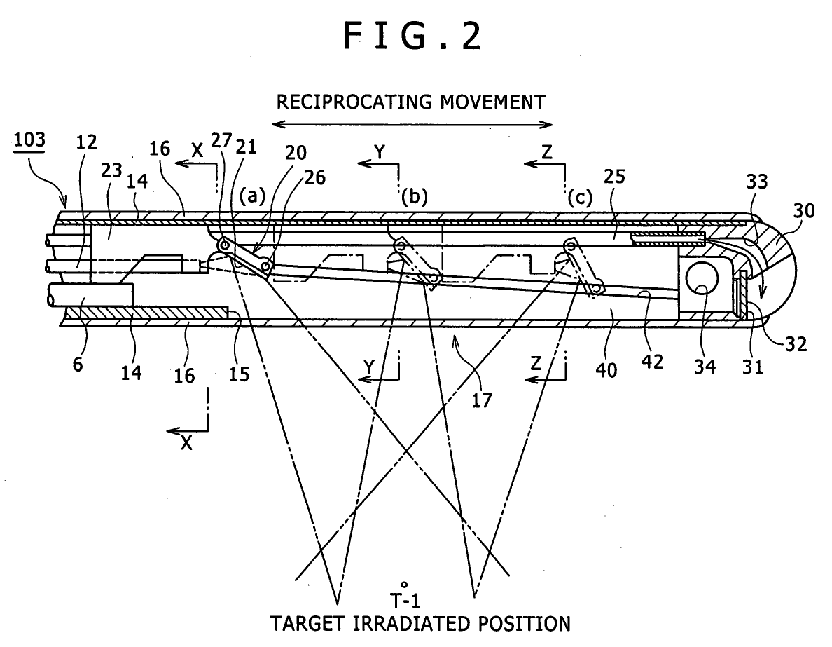

The laser beam irradiation portion 20 is coupled through a reciprocatingly movable member 23 (see FIG. 2) to a drive unit 150 disposed on the proximal end of the applicator 110. When the reciprocatingly movable member 23 is moved in the longitudinal direction of the insert portion-1-03 by the drive unit 150, the laser beam irradiation portion 20 is reciprocatingly moved in the directions indicated by the arrows.

The drive unit 150 has a cam mechanism (not shown) for converting rotary motion of a motor 188 into reciprocating motion. Therefore, when the motor 188 is energized, its rotary motion is converted by the cam mechanism into reciprocating motion that is transmitted to the reciprocatingly movable member 23, which moves the laser beam irradiation portion 20 in the longitudinal direction of the insert portion 103.

The applicator 110 has a plurality of lumens (not shown) defined longitudinally therein and communicating with the insert portion 103 for circulating a coolant. The lumens are connected respectively to a coolant supply tube 185 and a coolant return tube 186 which extend from a coolant circulator 104. The coolant is supplied through the coolant supply tube 185 to the insert portion 103 to cool the laser beam irradiation portion 20 for thereby preventing the laser beam irradiation portion 20 from being overheated, and also to cool the surface of the body cavity U, which is held in contact with the insert portion 103 through the wall of the insert portion 103, for thereby preventing a correct body tissue, which is heated by the applied laser beam, from being damaged.

The coolant circulator 104 supplies the coolant at a preset rate through the applicator 110 to the insert portion 103 based on a control signal from a controller 106. A coolant temperature regulator 105 that is coupled to the coolant circulator 104 heats or cools the coolant in the coolant circulator 104 to regulate the temperature of the coolant based on a control signal from a controller 106. The motor 188 is energized to rotate a preset rotational speed based on a control signal from a controller 106.

The controller 106 has a console 108 serving as an input unit, a display 107 for displaying input information and apparatus information, a control unit (not shown) for controlling various parts of the controller 106, a memory (not shown) for storing various items of information, and an input/output unit (not shown) for inputting and outputting various items of information.

The coolant is supplied from the coolant circulator 104 through the coolant supply tube 185 to the insert portion 103, the motor 188 is rotated, and a laser beam generator 102 is operated to treat, with heat, a prostatic target region T-1 (target point) to be irradiated with a laser beam.

A laser beam generated by the laser beam generator 102 is transmitted through the optical fiber 12 to the laser beam irradiation portion 20 in the insert portion 103, which reflects the laser beam through the laser beam irradiating window to the target region T-1. At this time, the laser beam irradiation portion 20 is reciprocatingly moved axially in the insert portion 103 in periodic cycles at a frequency ranging from 2 to 10 Hz, preferably 3 to 9 Hz, periodically changing the angle of irradiation. Since all the paths along which the reflected laser beam travels cross the target region T-1 at all times, the target region T-1 is continuously irradiated with the laser beam and generates a large amount of heat. Therefore, the target region T-1 is kept at a high temperature and can effectively be treated with heat. On the other hand, the surface layer of the body cavity U is intermittently irradiated with the laser beam, generating a small amount of heat, and is cooled by the coolant supplied to the insert portion 103. Consequently, the surface layer of the body cavity U is protected from and hence is not susceptible to the heat of the laser beam.

[Insert Portion (FIGS. 2, 3, and 4)]

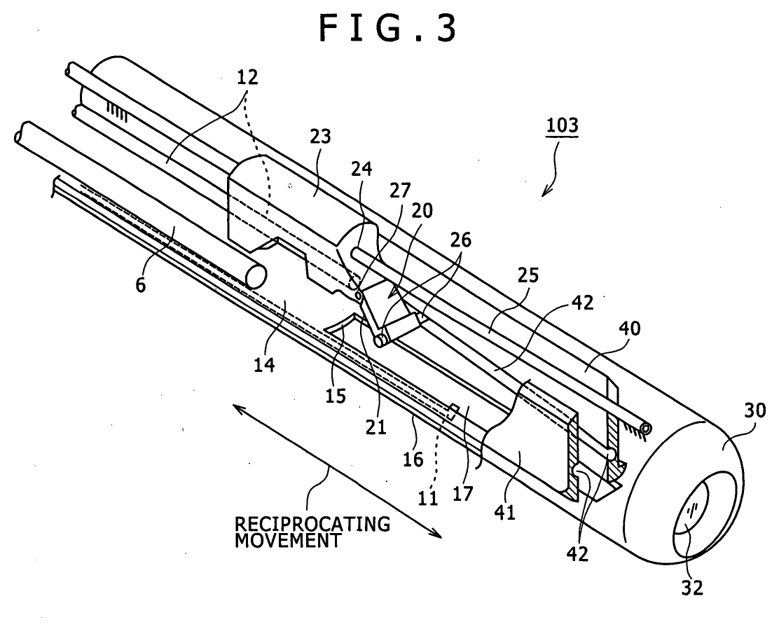

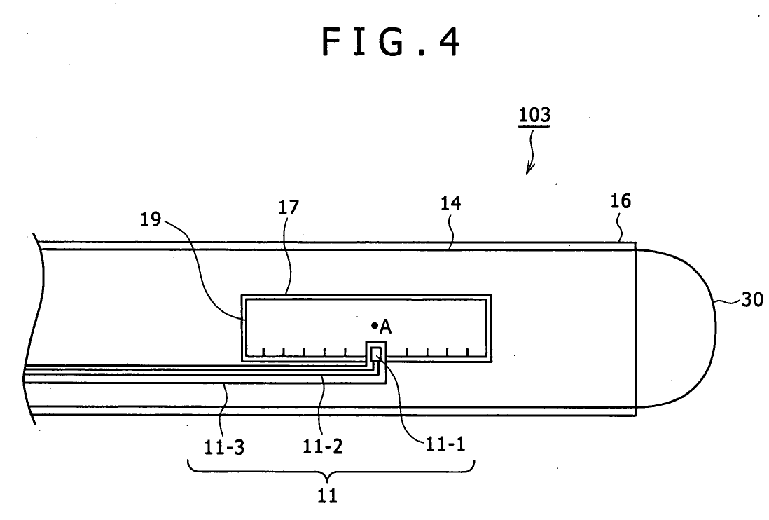

The insert portion 103 will be described in greater detail below. FIG. 2 shows the insert portion 103 in longitudinal cross section, FIG. 3 shows an internal structure of the insert portion 103, and FIG. 4 shows a temperature sensor disposed on a hollow cylinder 14.

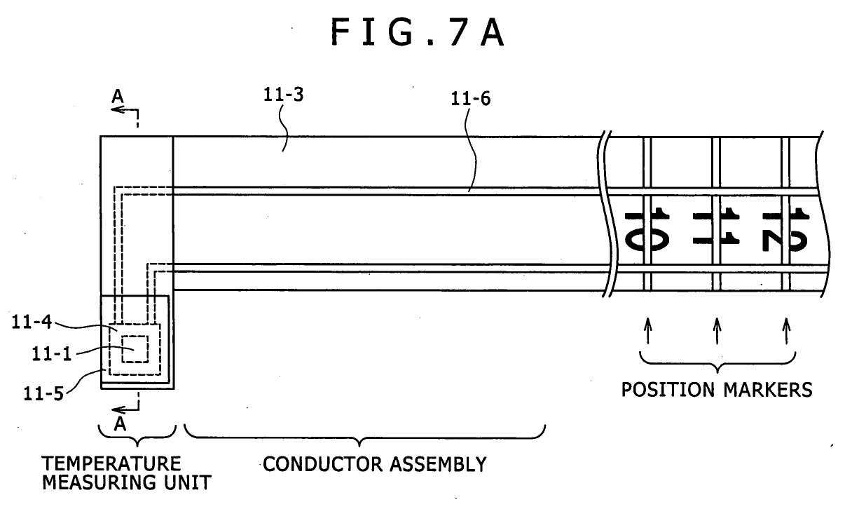

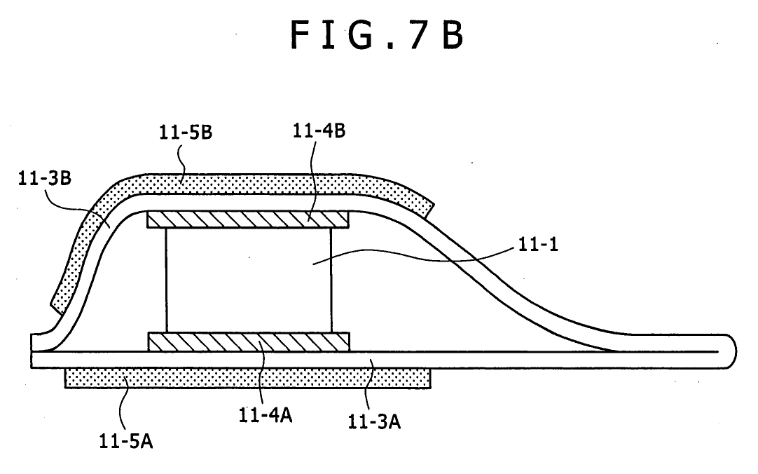

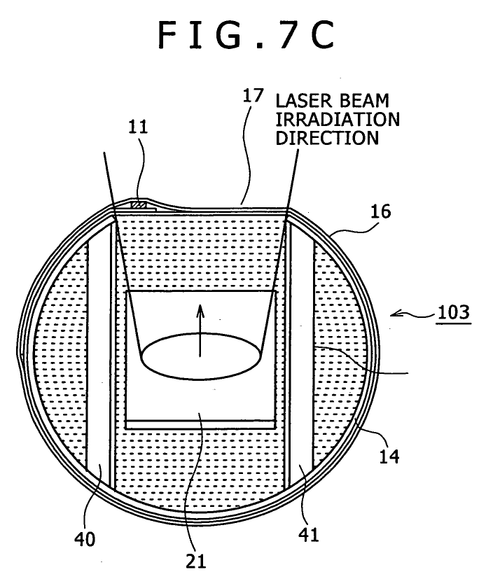

The insert portion 103 includes an elongate hollow cylinder 14 made of a hard pipe material such as stainless steel or the like, with an opening 15 defined in a side wall of the hollow cylinder 14. A graduated window seal is applied over the opening portion 15, providing a laser beam irradiating window 17. A temperature sensor 11 is mounted on the hollow cylinder 14. As shown in FIGS. 7A and 7B, the temperature sensor 11 includes a temperature measuring unit mounted on a thin-film substrate 11-3 and including a temperature measuring element 11-1 and electrodes 11-4A, 11-4B, and a conductor assembly mounted on the thin-film substrate 11-3 and including conductors 11-6. The hollow cylinder 14 has its outer circumferential surface covered entirely or partly with an outer tube 16, which is highly permeable to the laser beam. A cap 30 is sealingly fixed to the distal end of the hollow cylinder 14. The cap 30 has an optically transparent front window 32 for observing a forward region when the insert portion 103 is inserted into the body cavity U.

The insert portion 103 houses therein a pair of walls 40, 41 spaced laterally from each other, defining an inner space therebetween in the insert portion 103. The insert portion 103 also houses therein the laser beam irradiation portion 20 with the reflecting surface 21, the reciprocatingly movable member 23, a monorail pipe 25, nonparallel grooves 42, an endoscope 6, and coolant lumens. The reciprocatingly movable member 23 supports the laser beam irradiation portion 20. The monorail pipe 25 has the reciprocatingly movable member 23, which is reciprocatingly movable in the longitudinal direction of the insert portion 103. The nonparallel grooves 42 are defined in the respective walls 40, 41 for changing the angle of the laser beam irradiation portion 20 so that the laser beam reflected by the laser beam irradiation portion 20 is applied to the target region at all times. The endoscope 6 observes the living tissue. The laser beam irradiation portion 20 is rotatably supported on a pair of pivots 27 fixed to respective left and right sides of the reciprocatingly movable member 23 that is fixed to the distal end of the optical fiber 12. The laser beam irradiation portion 20 has a pair of lugs 26 mounted on respective left and right sides thereof and slidably fitted respectively in the nonparallel grooves 42 defined in the walls 40, 41. The nonparallel grooves 42 extend out of parallel with the longitudinal axis of the insert portion 103.

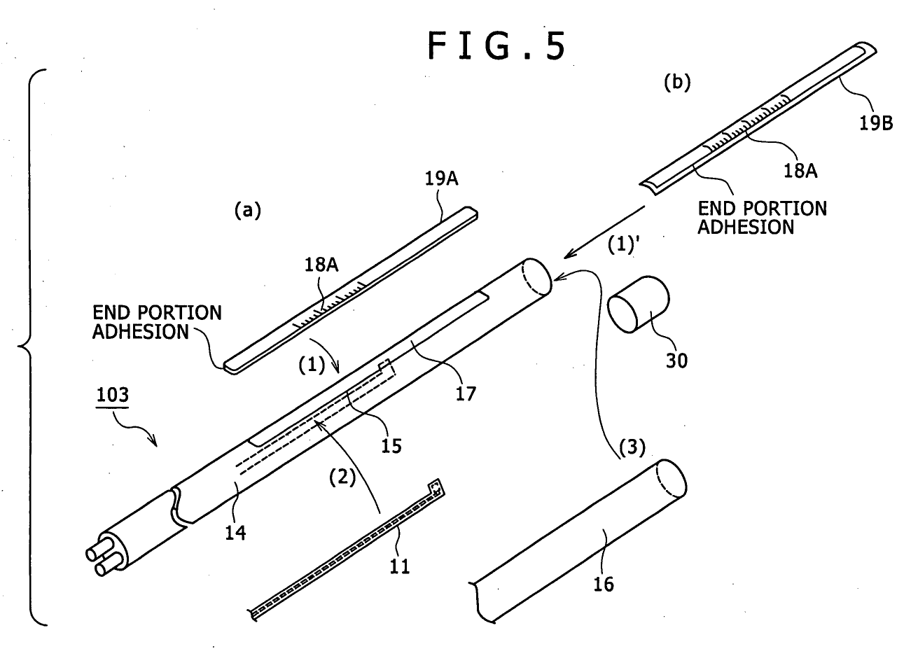

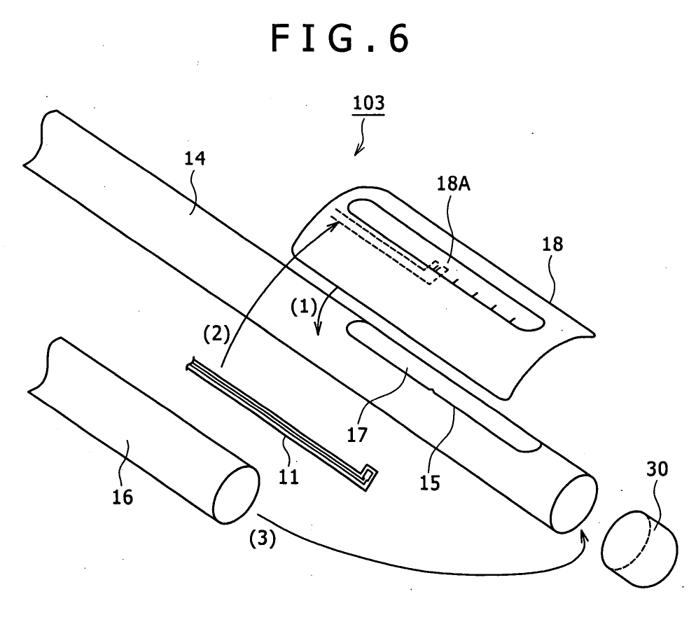

Major components of the insert portion 103 will be described below.

[Laser Beam Irradiating Window (FIGS. 5 and 6)]

FIGS. 5 and 6 are illustrative of a process of forming the laser beam irradiating window 17 using graduated glass strips 19A, 19B or a graduated window seal 18 and then placing the temperature sensor 11 on the hollow cylinder 14. The graduated glass strip 19A or 19B is produced by pressing a thin glass sheet into an arcuately curved glass strip with heat, and making a scale (graduation) 18A on the surface of the arcuately curved glass strip. The scale 18A is used to determine a position to be irradiated with a laser beam. The scale 18A is formed by printing or the like, at a position not obstructing the path of the laser beam and in a color that is not liable to absorb the laser beam.

The glass with scale (graduation) strip 19A or 19B is fixed in position over the opening 15. An adhesive is applied to an end of the glass with scale strip 19A, and the glass with scale strip 19A is fitted into the opening 15 from above and bonded to the hollow cylinder 14, as indicated at (1) in (a) of FIG. 5. Alternatively, an adhesive is applied to an end of the glass with scale strip 19B and the glass with scale strip 19B is inserted axially into the hollow cylinder 14, as indicated at (1)′ in (b) of FIG. 5, after which the glass with scale strip 19B is fitted into the opening 15 within the hollow cylinder 14 and bonded to the hollow cylinder 14.

FIG. 7A is a front elevational view showing a structure of the temperature sensor, and FIG. 7B is a cross-sectional view taken along line A-A of FIG. 7A. Structural details and features of the temperature sensor 11 will be described below with reference to FIGS. 7A and 7B.

As shown in FIG. 7A, the temperature sensor 11 is constructed of the temperature measuring unit and the conductor assembly. The conductor assembly includes the thin-film substrate 11-3 and the two conductors 11-6. The thin-film substrate 11-3 is made of an insulating material such as polyimide, nylon, polyethylene, PET, or the like. The two conductors 11-6 are mounted on the thin-film substrate 11-3 and each in the form of a strip of a conductive material. The thin-film substrate 11-3 has a plurality of position (depth) markers printed thereon for the user to easily read the length of the temperature sensor 11, which has been inserted into a living body. The thin-film substrate 11-3 includes a thin film having a thickness in the range from 10 to 40 μm, preferably from 15 to 25 μm, and can flexibly be bent. As shown in FIG. 7B, the temperature measuring unit has the temperature measuring element 11-1, such as a thermistor, disposed centrally therein, and the electrodes 114B, 114A mounted respectively on the upper and lower surfaces of the temperature measuring element 11-1. Thin-film substrates 11-3B, 11-3A are disposed respectively on the upper and lower surfaces of the electrodes 11-4B, 11-4A. Laser beam shield plates 11-5B, 11-5A are disposed respectively on the upper and lower surfaces of the thin-film substrates 11-3B, 11-3A.

[First Feature of Temperature Sensor (Thickness)]

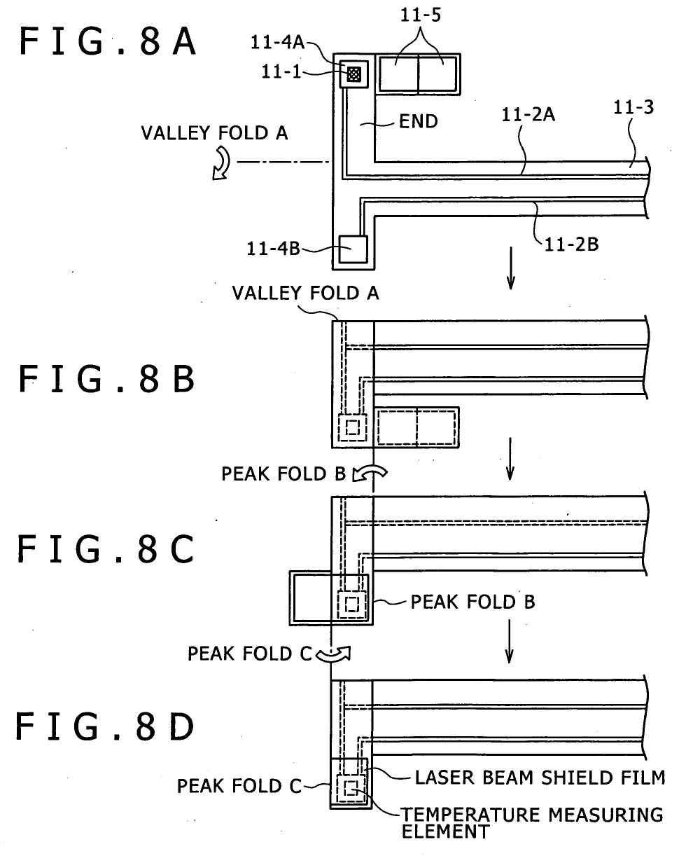

A pressing electrode according to a second feature of the temperature sensor 11 will be described below. Prior to describing the pressing electrode, a process of assembling the temperature measuring unit of the temperature sensor 11 will first be described below with reference to FIGS. 8A through 8D. FIG. 8A shows one example of the previous state assembled into the temperature sensor 11. The temperature sensor includes conductors 11-2A, 11-2B, electrodes 11-4A, 11-4B, and a laser beam shield film 11-5, which are formed by etching or the like on a thin-film substrate 11-3 shaped as shown in FIG. 8A. The conductors 11-2A, 11-2B, the electrodes 114-A, 11-4B, and the laser beam shield film 11-5 are formed of one conductive material, e.g., copper, on the thin-film substrate 11-3 by etching or the like. A temperature measuring element 11-1 is bonded to the electrode 11-4A by a conductive adhesive. The electrodes 11-4A, 11-4B and the surface of the laser beam shield film 11-5 may be covered with evaporated gold. The conductors 11-2A, 11-2B need to be covered with a printed resist layer or another cover layer such as of polyimide, nylon, polyethylene, PET, or the like so as to prevent a short circuit therebetween.

The reflecting surface 21 of the laser beam irradiation portion 20 disposed in the insert portion 103 will be described below. The reflecting surface 21 constitutes part of the laser beam irradiation portion 20, and has a smooth surface for reflecting the laser beam emitted from the distal end of the optical fiber 12 through the laser beam irradiating window 17 to the target region T-1.

[Monorail Pipe (FIG. 2)]

As shown in FIG. 2, the monorail pipe 25 is a hollow pipe for passing a cleaning medium such as a cleaning liquid, a cleaning gas, or the like therethrough. The monorail pipe 25 allows the reciprocatingly movable member 23 to move therealong in the longitudinal direction of the insert portion 103, and also serves as a pipe for supplying a cleaning medium such as a cleaning liquid, a cleaning gas, or the like from a cleaning unit (not shown) to the front window 32 of the insert portion 103 when the front window 32 is dirtied.

The reciprocatingly movable member 23 serves to change the direction of the applied laser beam depending on the irradiating position thereof when the reciprocatingly movable member 23 moves on the monorail pipe 25 in the directions indicated by the arrows, i.e., in the longitudinal directions of the applicator 110, e.g., from the position (a) to the position (b) to the position (c) to the position (b) to the position (a). Therefore, the direction of the applied laser beam and the irradiating position thereof can continuously be changed to control the laser beam to irradiate the target position at all times.

The reciprocatingly movable member 23 supports the laser beam irradiation portion 20 for reciprocating movement therewith. The reciprocatingly movable member 23 is positioned on one end of the laser beam irradiation portion 20, and the lugs 26 are positioned on the opposite end of the laser beam irradiation portion 20. The laser beam irradiation portion 20 is mounted on the reciprocatingly movable member 23 by the pivots 27 for free angular movement with respect to the reciprocatingly movable member 23 for thereby allowing the angle of the reflecting surface 21 to be changed with respect to the reciprocatingly movable member 23. The lugs 26 are fitted in the respective nonparallel grooves 42 that are defined in the inner surfaces of the walls 40, 41 disposed in the insert portion 103.

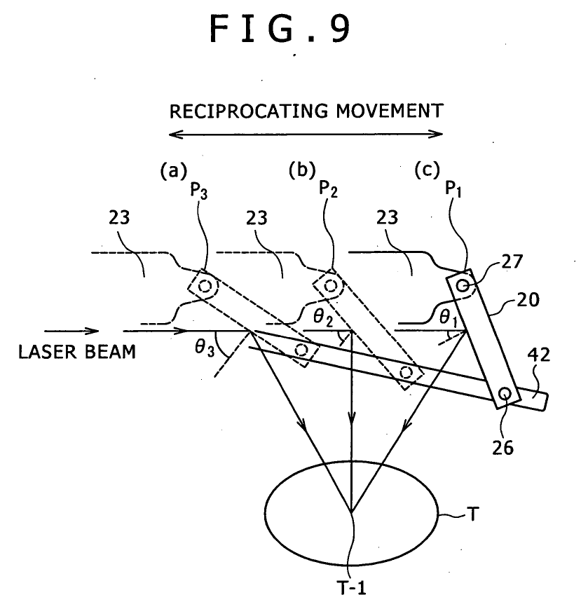

The reciprocatingly movable member 23 is coupled to the drive unit 150 (see FIG. 1), which is disposed on the proximal end of the applicator 110. When the reciprocatingly movable member 23 is slid on the monorail pipe 25 by the drive unit 150, the laser beam irradiation portion 20 is reciprocatingly moved in the longitudinal directions of the insert portion 103. When the laser beam irradiation portion 20 is axially moved by the reciprocatingly movable member 23 that travels on the monorail pipe 25, the laser beam irradiation portion 20 is caused by the nonparallel grooves 42 to change the angle of the reflecting surface 21.

[Direction of Applied Laser Beam (FIG. 9)]

FIG. 9 is illustrative of the relationship between the movement of the laser beam irradiation portion 20 and the direction of the applied laser beam reflected by the laser beam irradiation portion 20.

As shown in FIG. 9, the distance between the reciprocatingly movable member 23 and the nonparallel grooves 42 in the position P2 (the position (b)) is shorter than the distance between the reciprocatingly movable member 23 and the nonparallel grooves 42 in the position P1 (the position (c)). Therefore, when the reciprocatingly movable member 23 moves from the position P1 (the position (c)) to the position P2 (the position (b)), the lugs 26 of the laser beam irradiation portion 20 are lifted as they move along the nonparallel grooves 42. The angle of tilt of the laser beam irradiation portion 20 is adjusted. That is to say, the angle of tilt of the laser beam irradiation portion 20 with respect to the monorail pipe 25 is reduced. Similarly, when the reciprocatingly movable member 23 moves from the position P2 (the position (b)) to the position P3 (the position (a)), the angle of tilt of the laser beam irradiation portion 20 with respect to the monorail pipe 25 is further reduced.

The laser beam reflected by the laser beam irradiation portion 20 is applied to the target region T-1 (target point) of the prostate T at all times when the reciprocatingly movable member 23 is in the positions P1 through P3. Therefore, the laser beam continuously irradiates the target region T-1, and intermittently irradiates other tissue regions such as the surface layer of the body cavity U. The target region T-1 that is continuously irradiated with the laser beam generates a large amount of heat and reaches a desired high temperature, whereas the surface layer of the body cavity U, which is intermittently irradiated with the laser beam, generates a small amount of heat and is not heated to a high temperature. Consequently, only the target region T-1 and its surrounding regions are selectively heated by the laser beam for treatment with heat.

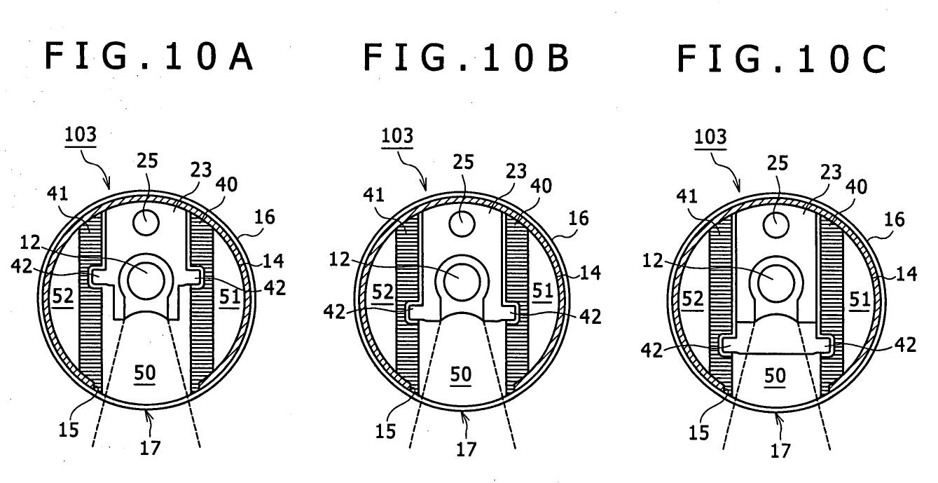

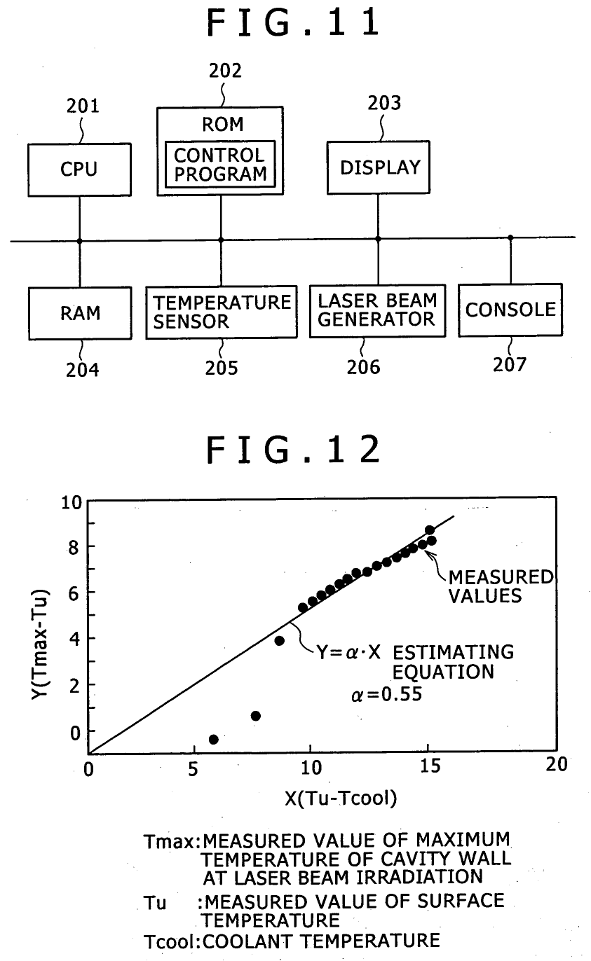

The laser beam irradiation portion 20 for reflecting the laser beam is reciprocatingly moved on and along the monorail pipe 25 in periodic cycles at a frequency ranging from 2 to 10 Hz, preferably 3 to 9 Hz, in the longitudinal direction of the insert portion 103 while changing its angle.

[Nonparallel Grooves (FIG. 10)]

Structural details of the nonparallel grooves 42 will be described below with reference to FIGS. 10A through 10C.

FIGS. 10A through 10C are transverse cross-sectional views of the insert portion 103 respectively at the positions (a), (b), and (c) in FIG. 2, showing the different vertical positions of the nonparallel grooves 42 defined in the walls 40, 41 at the respective positions (a), (b), and (c).

As shown in FIGS. 10A through 10C, the two laterally spaced walls 40, 41 are disposed in the insert portion 103. The monorail pipe 25 for delivering the cleaning medium therethrough, the optical fiber 12 for guiding the laser beam, and a coolant inlet lumen 50 for delivering the coolant to the distal end of the insert portion 103 are disposed between the walls 40, 41.

Coolant outlet lumens 51, 52 for returning the coolant from the distal end of the insert portion 103 to the coolant circulator 104 are disposed between the circumferential wall of the hollow cylinder 14 and the walls 40, 41.

The position of the nonparallel grooves 42 at the position in FIG. 10A is higher than the position of the nonparallel grooves 42 at the position in FIG. 10B. Therefore, the reflecting angle θ₃ of the laser beam irradiation portion 20 for reflecting the laser beam at the position (a) in FIG. 9 is greater than the reflecting angle θ₂ of the laser beam irradiation portion 20 for reflecting the laser beam at the position (b) in FIG. 9.

Likewise, the position of the nonparallel grooves 42 at the position in FIG. 10B is higher than the position of the nonparallel grooves 42 at the position in FIG. 10C. Therefore, the reflecting angle θ₂ of the laser beam irradiation portion 20 for reflecting the laser beam at the position (b) in FIG. 9 is greater than the reflecting angle θ₁ of the laser beam irradiation portion 20 for reflecting the laser beam at the position (c) in FIG. 9.

Consequently, the laser beam reflected by the laser beam irradiation portion 20 is concentrated on the target region T-1 at all times based on the different vertical positions of the nonparallel grooves 42.

[Temperature Control System (FIG. 11)]

A temperature control system of the energy irradiating medical apparatus will be described below.

FIG. 11 shows in block form a control circuit of the energy irradiating medical apparatus. As shown in FIG. 11, the control circuit includes a CPU 201, a ROM 202 for storing a control program that is executed by the CPU 201, a display 203, a RAM 204 for storing various data, a temperature sensor 205, a laser beam generator 206, and a console 207.

Operation of the control circuit will be described below. The console 207 includes a keyboard or the like. The user enters from the console 207 a signal for starting various processes for displaying a maximum surface temperature, displaying a deep region temperature, determining an incorrect irradiation timing, and controlling a laser beam output level. When the CPU 201 receives a command for executing the various processes, the CPU 201 operates according to the control program stored in the ROM 202 to receive measured values of the surface temperature from the temperature sensor 11 in the insert portion 103, store the measured values in the RAM 204, and control the laser beam generator 206 and the display 203 based on the measured values for displaying a maximum surface temperature, displaying a deep region temperature, determining an incorrect irradiation timing, and controlling a laser beam output level.

[Process of Estimating Maximum Cavity Wall Temperature (FIGS. 12 Through 14)]

A process of estimating a maximum cavity wall temperature upon laser beam irradiation from measured values of the surface temperature, which are produced by the temperature sensor 11 in the insert portion 103 when the doctor treats an affected region with the energy irradiating medical apparatus 10, will be described below.

First, measuring conditions will be described below. The temperature sensor 11 is disposed at a circumferential end of the laser beam irradiating window 17 shown in FIG. 4 in its longitudinally central area, and measures a surface temperature Tu upon laser beam irradiation. A maximum cavity wall temperature Tmax upon laser beam irradiation is always observed at a central point A in the laser beam irradiating window 17 shown in FIG. 4. The maximum cavity wall temperature Tmax is measured by a temperature sensor, separate from the temperature sensor 11, positioned at the central point A. Tcool represents the temperature of the coolant for cooling the interior of the insert portion 103.

FIG. 12 shows the correlation between measured values of the surface temperature and measured values of the maximum cavity wall temperature upon laser beam irradiation. In FIG. 12, the horizontal axis represents X=Tu−Tcool and the vertical axis Y=Tmax−Tcool. Solid dots in FIG. 12 show measured values. In FIG. 12, a linear curve Y=α·X represents an estimating equation determined by linearly approximating the measured values, where α=0.55. As it is understood from FIG. 12 that the surface temperature Tu and the maximum cavity wall temperature Tmax upon laser beam irradiation satisfy the following equation:
Tmax=Tcool+(1+α)(Tu−Tcool) (1)
the maximum cavity wall temperature Tmax can be estimated from the surface temperature Tu upon laser beam irradiation according to the equation (1).

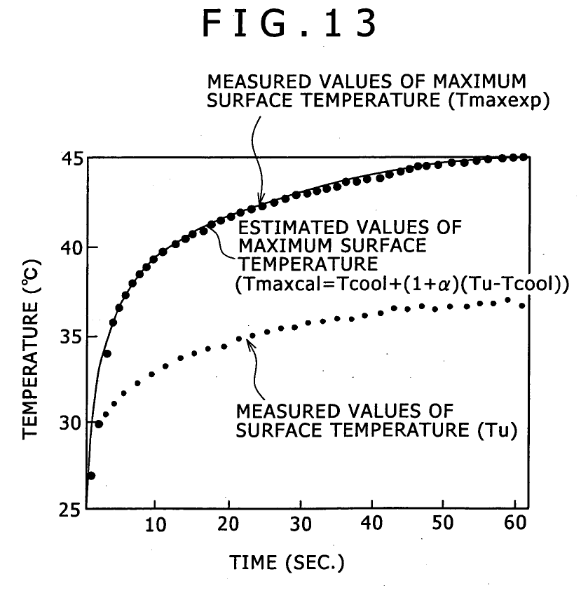

FIG. 13 shows estimated values (Tmaxcal) of the maximum cavity wall temperature obtained from the surface temperature Tu upon laser beam irradiation according to the equation (1), and measured values (Tmaxexp) of the maximum cavity wall temperature. Since the measured and estimated values of the maximum cavity wall temperature at desired times agree with each other, the maximum cavity wall temperature Tmax can be estimated from the surface temperature Tu upon laser beam irradiation according to the equation (1).

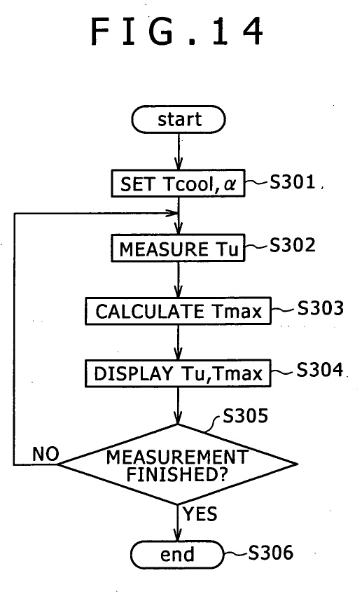

Based on the above experimental results, a control program for calculating the maximum cavity wall temperature Tmax from the surface temperature Tu upon laser beam irradiation is produced and stored in the ROM 202. FIG. 14 shows a process carried out by the CPU 201 according to the control program. The process is started when the doctor enters an execution command and initial values for executing the control program from the console when the doctor treats an affected region with the energy irradiating medical apparatus.

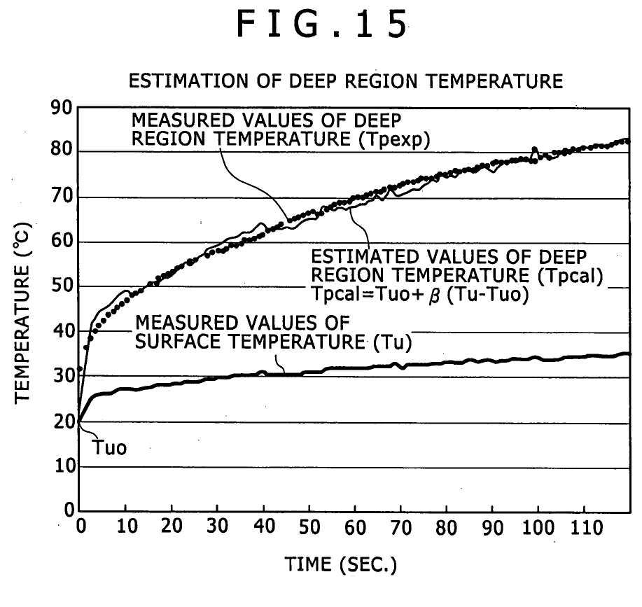

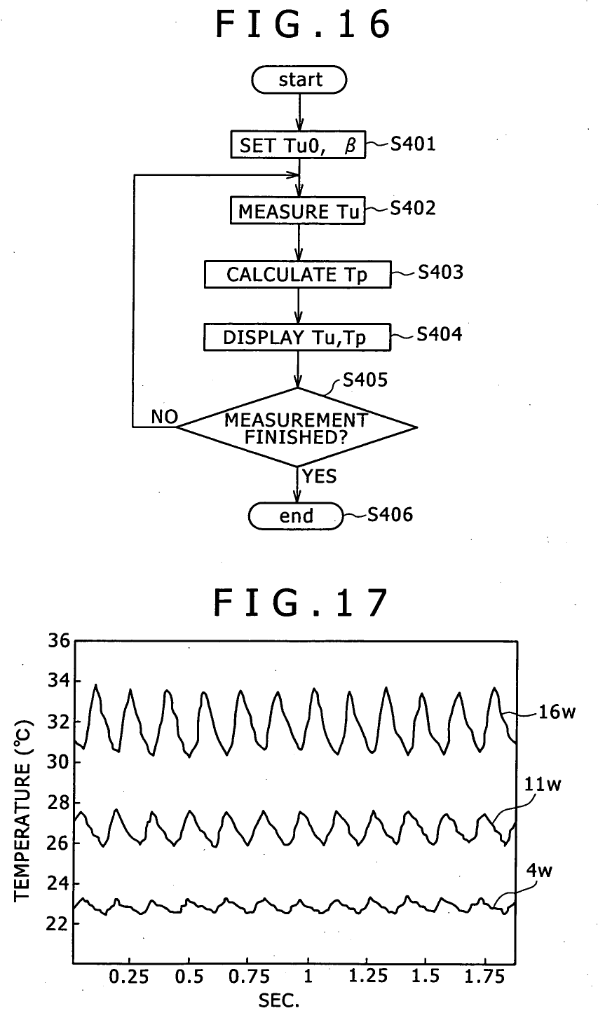

In step S301, Tcool and α are set. In step S302, the surface temperature Tu is measured. In step S303, the maximum cavity wall temperature Tmax is calculated according to the equation (1). In step S304, the measured surface temperature Tu and the calculated maximum cavity wall temperature Tmax are displayed on the display. If a next measuring cycle is to be performed in step S305, then control goes back to step S302, and the above process is repeated. If the present measuring cycle is to be finished in step S305, then control goes to step S306, putting the process to an end.

[Process of Estimating Deep Region Temperature (FIGS. 15 and 16)]

A process of estimating a deep region temperature in a living body upon laser beam irradiation from measured values of the surface temperature, which are produced by the temperature sensor 11 in the insert portion 103 when the doctor treats an affected region with the energy irradiating medical apparatus 10, will be described below.

First, measuring conditions will be described below. The temperature sensor 11 is disposed at a circumferential end of the laser beam irradiating window 17 shown in FIG. 4 in its longitudinally central area, and measures a surface temperature Tu upon laser beam irradiation. A deep region temperature Tp upon laser beam irradiation is a point B. The point B is located a depth of 1 cm directly below the surface of the living tissue that is held in contact with a central point A in the laser beam irradiating window 17 shown in FIG. 4. The temperature sensor is inserted into the point B, and Tp is measured. Tu0 represents an initial value of the temperature measured by the temperature sensor 11.

A process that is the same as the process described above with reference to FIG. 12 is carried out. As it is understood that the surface temperature Tu and the deep region temperature Tp upon laser beam irradiation satisfy the following equation:
Tp=Tu0+α(Tu−Tu0) (2)
the deep region temperature Tp can be estimated from the surface temperature Tu upon laser beam irradiation according to the equation (2).

FIG. 15 shows estimated values (Tpcal) of the deep region temperature obtained from the surface temperature Tu upon laser beam irradiation according to the equation (2), and measured values (Tpexp) of the deep region temperature. Since the measured and estimated values of the deep region temperature at desired times agree with each other, the deep region temperature Tp can be estimated from the surface temperature Tu upon laser beam irradiation according to the equation (2). (α=4.2)

Based on the above experimental results, a control program for calculating the deep region temperature Tp from the surface temperature Tu upon laser beam irradiation is produced and stored in the ROM 202. FIG. 16 shows a process carried out by the CPU 201 according to the control program. The process is started when the doctor-enters an execution command and initial values for executing the control program from the console when the doctor treats an affected region with the energy irradiating medical apparatus.

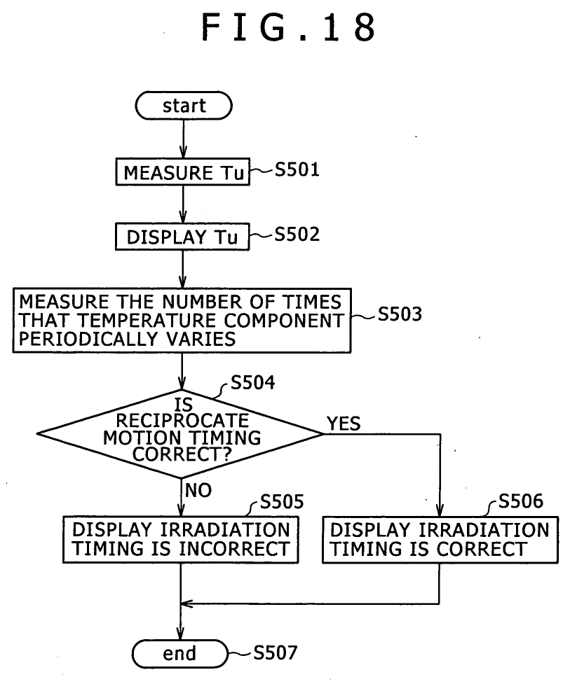

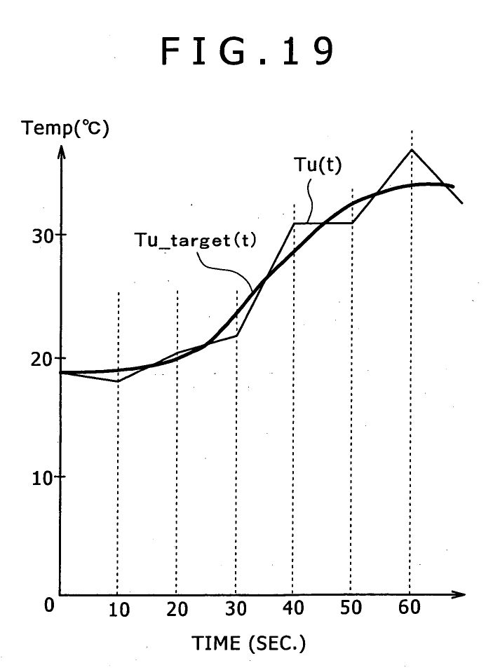

In step S401, Tu0 and β are set. In step S402, the surface temperature Tu is measured. In step S403, the deep region temperature Tp is calculated according to the equation (2). In step S404, the measured surface temperature Tu and the calculated deep region temperature Tp are displayed on the display. If a next measuring cycle is to be performed in step S405, then control goes back to step S402, and the above process is repeated. If the present measuring cycle is to be finished in step S405, then control goes to step S406, putting the process to an end. [Monitoring of reciprocating motion timing (FIGS. 17 and 18)]

A process of monitoring irradiation timing upon laser beam irradiation from measured values of the surface temperature, which are produced by the temperature sensor 11 in the insert portion 103 when the doctor treats an affected region with the energy irradiating medical apparatus 10, will be described below.

First, measuring conditions will be described below. The temperature sensor 11 is disposed at a circumferential end of the laser beam irradiating window 17 shown in FIG. 4 in its longitudinally central area, and measures a surface temperature Tu upon laser beam irradiation. The temperature sensor 11 used has laser beam shield plates uncovered.

FIG. 17 shows temperature changes caused in 2 seconds when a laser beam is applied at output levels of 4, 11, and 16 W and the laser beam irradiation portion 20 is reciprocatingly moved at a frequency of 6 Hz. When a laser beam having an output level of 16 W is applied, the measured temperature values are in a range between a lowest temperature of 30° C. and a highest temperature of 34° C., and periodically vary six times per second. When laser beams having other output levels are applied, the measured temperature values also periodically vary six times per second. This indicates that the laser beam irradiation portion 20 is repeatedly reciprocatingly moved six times per second, applying the laser beam correctly to the target region. Therefore, it is possible to determine whether or not the laser beam irradiation portion 20 is operating correctly by measuring the number of periodical temperature changes per unit period of time. For example, under the above conditions, the irradiation timing is determined as being correct if six periodical temperature changes per second are detected, and is determined as being incorrect if more than six periodical temperature changes per second or less than six periodical temperature changes per second are detected.

[Detection of Laser Beam Output Level]

The output level of a laser beam emitted from the laser beam generator can be measured from the range of temperature changes shown in FIG. 17. Specifically, the relationship between laser beam output levels and temperature changes may be stored in the ROM, and a laser beam output level may be calculated from a measured temperature change based on the stored relationship.

Based on the above experimental results, a control program for determining whether the irradiation timing is correct or not from the surface temperature Tu upon laser beam irradiation is produced and stored in the ROM 202. FIG. 18 shows a process carried out by the CPU 201 according to the control program. The process is started when the doctor enters an execution command and initial values for executing the control program from the console when the doctor treats an affected region with the energy irradiating medical apparatus.

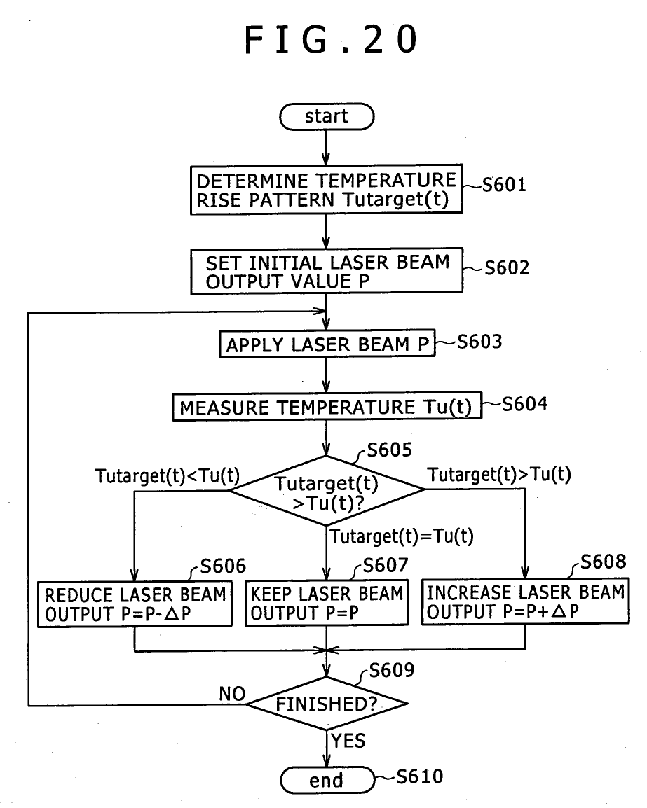

In step S501, the surface temperature Tu is measured for a certain period of time. In step S502, the measured surface temperature Tu is displayed. In step S503, the number of periodic temperature changes is measured in the above period of time from the measured values of the surface temperature Tu, and it is checked whether or not the measured number of periodic temperature changes agrees with a preset number of periodic temperature changes. If the measured number of periodic temperature changes agrees with the preset number of periodic temperature changes, then control goes to step S506 in which correct reciprocating motion timing is displayed. Then, control goes to step S507 to put the process to an end. If the measured number of periodic temperature changes does not agree with the preset number of periodic temperature changes, then control goes to step S505 in which incorrect reciprocating motion timing is displayed. Then, control goes to step S507 to put the process to an end.

[Control of Laser Beam Output Level (FIGS. 19 and 20)]

A process of controlling a laser beam output level upon laser beam irradiation based on measured values of the surface temperature, which are produced by the temperature sensor 11 in the insert portion 103 when the doctor treats an affected region with the energy irradiating medical apparatus 10, will be described below.

First, measuring conditions will be described below. The temperature sensor 11 is disposed at a circumferential end of the laser beam irradiating window 17 shown in FIG. 4 in its longitudinally central area, and measures a surface temperature Tu upon laser beam irradiation. FIG. 19 shows a preset temperature rise pattern Tutarget(t) of the surface temperature upon laser beam irradiation and measured values of the surface temperature Tu. A living tissue is heated according to the preset temperature rise pattern. It is necessary to change the laser beam output level upon laser beam irradiation from time to time. The laser beam output level is controlled by the CPU 201, which controls the laser beam generator 206, according to a predetermined control program based on the measured values of the surface temperature Tu. FIG. 20 shows a process carried out by the CPU 201 according to the control program. The process is started when the doctor enters an execution command and initial values for executing the control program from the console when the doctor treats an affected region with the energy irradiating medical apparatus.

In step S601, a temperature rise pattern Tutarget(t) of the surface temperature upon laser beam irradiation is determined. Specifically, the doctor selects a desired one of a plurality of preset temperature rise patterns, and the CPU 201 determines the temperature rise pattern based on a selection signal entered by the doctor. In step S602, an initial laser beam output level is set. In step S603, the target region is irradiated with a laser beam having the initial laser beam output level. In step S604, the surface temperature T(t) upon laser beam irradiation is measured. In step S605, the measured surface temperature T(t) is compared with the temperature rise pattern Tutarget(t). If Tutarget(t)<Tu(t) in step S605, then control goes to step S606 in which the laser beam output level P is changed to P−ΔP, after which control goes to step S609. If Tutarget(t)=T(t) in step S605, then control goes to step S607 in which the laser beam output level P is not changed, but maintained, after which control goes to step S609. If Tutarget(t)>T(t) in step S605, then control goes to step S608 in which the laser beam output level P is changed to P+ΔP, after which control goes to step S609. If a next output level controlling cycle is to be performed in step S609, then control goes back to step S603, and the above process is repeated. If the present output level controlling cycle is to be finished in step S609, then control goes to step S610, putting the process to an end.

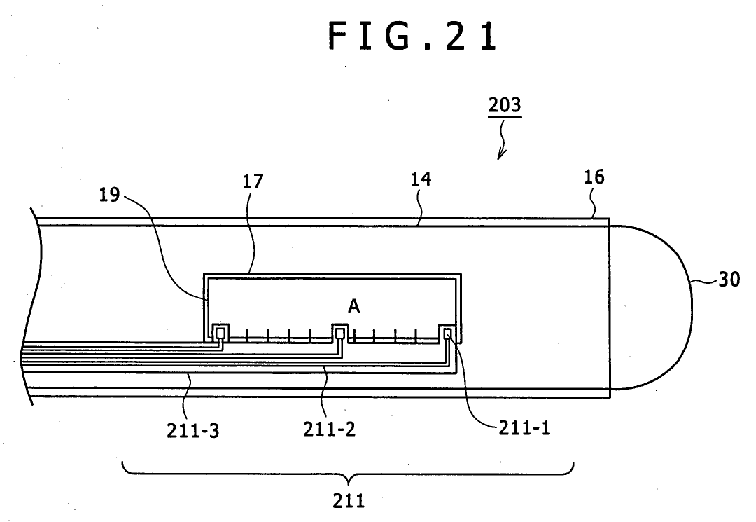

In the above embodiment, the single temperature sensor 11 is disposed in the insert portion 103 as shown in FIG. 4. However, a plurality of temperature sensors may be disposed on the insert. FIG. 21 shows three independent temperature sensors disposed on one thin-film substrate. The temperature sensors shown in FIG. 21 can be manufactured according to a process based on the process shown in FIGS. 8A through 8D. Therefore, the process of manufacturing the temperature sensors shown in FIG. 21 will not be described in detail below. The plural temperature sensors shown in FIG. 21 make it possible to measure more accurately temperature changes in a living tissue as it is treated with heat.

The energy irradiating medical apparatus according to the present invention should preferably be used to treat a prostate with heat to cure a prostatic disease such as a prostatic hypertrophy, a prostatic cancer, or the like while reducing damage to a correct living tissue, such as the urethra, the rectum, or the like, that is positioned closely to the prostate.

As described above, the temperature measuring unit of the temperature sensor according to the embodiment of the present invention described above has the electrodes disposed on the upper and lower surfaces of the temperature measuring element, the thin-film substrates disposed on the upper and lower surfaces of the electrodes, and the laser beam shield plates disposed on the upper and lower surfaces of the thin-film substrates. The electrode 114A is bonded to the temperature measuring element by the conductive adhesive, and the electrode 11-4B is not bonded to the temperature measuring element by the conductive adhesive. When the temperature sensor is bonded to the hollow cylinder of the insert, the temperature measuring unit is curved along the surface of the hollow cylinder, tending to develop tensile stresses in the electrode 11-4B. At this time, the electrode 114B, which is not bonded to the temperature measuring element, is positionally displaced depending on the developed tensile stresses, allowing the temperature sensor to be adjusted in length. Consequently, the temperature sensor is prevented from being broken or damaged. Therefore, the energy irradiating medical apparatus according to the present invention is capable of accurately measuring the temperature of a living tissue as it is treated with heat, though the energy irradiating medical apparatus is simple in structure and inexpensive to manufacture. Therefore, the doctor who operates the energy irradiating medical apparatus can correctly monitor the temperature of the living tissue as it is treated with heat to cure a prostatic hypertrophy, for example, and hence can treat the living tissue with greater safety.

A temperature sensor (thin thermistor) for use in an energy irradiating medical apparatus according to the first embodiment has useful application in a variety of respects. For example, when an insert portion having a laser beam irradiating window for applying a laser beam is inserted from a lumen such as urethra and the laser beam is applied from the laser beam irradiating window of the insert portion to the deep region of a living tissue to treat benign prostatic hypertrophy, for example, with heat, the energy irradiating medical apparatus can accurately measure the temperature of the living tissue being treated with heat, for increasing the curative effect. The thin thermistor has a temperature measuring unit including a temperature measuring element made of a transition-metal oxide containing Mn, Co, Ni, or Fe. The temperature measuring element has a thickness of about 200 μm which makes itself small in size. The temperature measuring element has a measuring area of 0.09 mm², for example, and is suitable for measuring a local temperature (spot temperature). As shown in FIG. 7B, the temperature measuring element is shielded from light by a laser beam shield plate. When the temperature measuring element is installed in a peripheral region in the laser beam irradiating window for intermittently detecting a laser beam, the temperature measuring element is capable of accurately measuring the temperature of the surface of the lumen. The temperature measuring unit has a response speed of about 200 msec., and is suitable for measuring the temperature of the surface of the lumen by intermittently detecting the laser beam when the living tissue is treated with heat by the laser beam irradiation portion that reciprocatingly moves at a frequency ranging from 3 to 10 Hz.

The energy irradiating medical apparatus can estimate a maximum temperature of the surface of the lumen and a temperature (laser beam irradiation target temperature) of the deep region heated by being irradiated with the laser beam, from the measured temperature of the surface of the lumen. Therefore, the energy irradiating medical apparatus can continuously estimate and display, on a display portion, time-dependent changes of the maximum temperature of the surface of the lumen and the temperature of the deep region. The energy irradiating medical apparatus can be controlled so that when the measured temperature exceeds a preset temperature, the energy irradiating medical apparatus issues a light or sound warning to prompt the operator to pay attention or stops applying the laser beam. Therefore, the living tissue is prevented from being irreversibly damaged due to denaturization of protein (the living tissue is irreversibly damaged if exposed to the temperature of 55° C. for about 20 seconds, the temperature of 50° C. for about 5 minutes, and the temperature of 48° C. for about 10 minutes). The doctor monitors the maximum temperature of the surface of the lumen displayed on the display portion. Thus, the doctor can change the irradiation condition of the laser beam at the heat treatment so that the urethra is prevented from being damaged. The doctor can monitor the effectiveness of the heat treatment or control the application of the laser beam depending on the temperature of the deep region by monitoring the temperature of the deep region displayed on the display portion. For example, if the temperature of the deep region is too low, the doctor can intensify the application of the laser beam, and if the temperature of the deep region has reached a target temperature, the doctor can stop applying the laser beam.

Consequently, the energy irradiating medical apparatus according to the first embodiment is of a structure that is simple and inexpensive to manufacture, and which is capable of safely treating a living tissue with heat by accurately measuring the temperature of the living tissue while it is being treated with heat. Since the temperature measuring unit is thin, the insert portion may be reduced in size to alleviate the pain from the patient when the insert portion is inserted into the patient. As the temperature measuring element does not need to be connected to two leads and placed in a tangle-free manner in a protective tube unlike the structure in related art, the insert portion can be reduced in size. Since the temperature measuring element is not disposed in the insert portion, it is less affected by cooling water and can measure the temperature of the surface of the living body with high accuracy. Since the temperature sensor is not required to directly thrust into a living tissue to measure the temperature of the living tissue, the living tissue is prevented from being damaged by thrusting thereinto and also from a side effect due to an infectious disease.

According to the first embodiment described above, the energy irradiating medical apparatus 10 employs a thin thermistor as a temperature sensor. According to a second embodiment, an energy irradiating medical apparatus 110 employs a temperature sensor including a temperature measuring element in the form of a thin-film metal resistor. In the following description, the thin-film metal resistor is made of aluminum. However, the thin-film metal resistor is not limited to being made of aluminum, but may be made of Pt, W, Ni, Co, Ag, Au, Cu, or the like, for example. A temperature sensor whose thin-film metal resistor is made of aluminum is referred to as a temperature sensor (aluminum sensor). The energy irradiating medical apparatus 10 according to the first embodiment and the energy irradiating medical apparatus 110 according to the second embodiment have similar structural details except that they have different temperature sensors. Those parts of the energy irradiating medical apparatus 110 according to the second embodiment which are identical to those of the energy irradiating medical apparatus 10 according to the first embodiment are denoted by identical reference characters, and will not be described below. The detailed description below will be directed primarily at those parts of the energy irradiating medical apparatus 110 according to the second embodiment which are different from those of the energy irradiating medical apparatus 10 according to the first embodiment.

[Insert Portion (FIGS. 2, 3, and 22)]

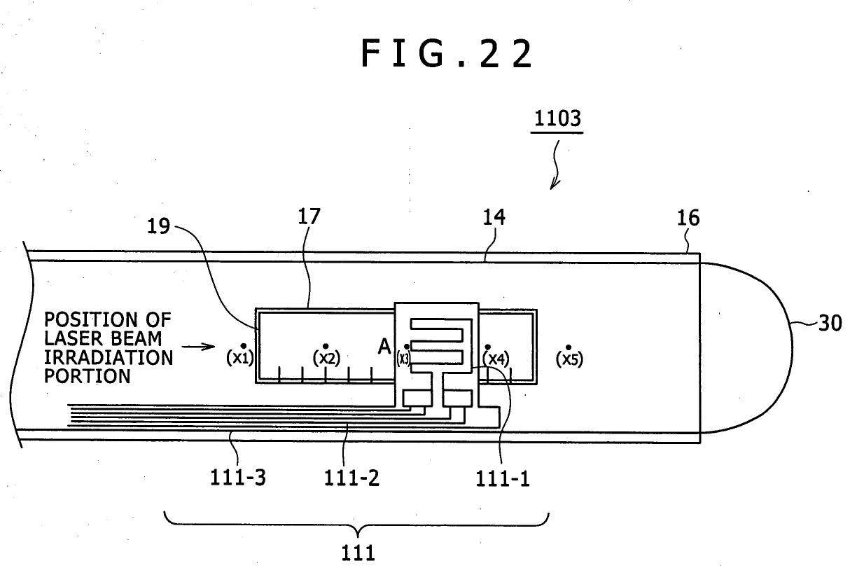

The energy irradiating medical apparatus 110 according to the second embodiment for treating benign prostatic hypertrophy with heat has a system arrangement which is identical to that shown in FIG. 1, except a temperature sensor and its control. The description of the features associated with the arrangement shown in FIG. 1 which have already been described will be omitted, and the insert portion 1103 will be described below. The insert portion 1103 is the same general cross-sectional view shown in FIG. 2, and its inner structure is the same general perspective view shown in FIG. 3. A temperature sensor (aluminum sensor) disposed on the hollow cylinder 14 is shown in FIG. 22.

As shown in FIG. 22, the insert portion 1103 includes an elongate hollow cylinder 14 made of a hard pipe material such as stainless steel or the like, with an opening portion 15 defined in a side wall of the hollow cylinder 14. A window seal with scale 18 is applied over the opening portion 15, or a graduated glass strip 19 is set in the opening portion 15, providing a laser beam irradiating window 17. A temperature sensor (aluminum sensor) 111 including a temperature measuring unit 111-1, a conductor assembly 111-2, and a thin-film substrate 111-3 is mounted on the hollow cylinder 14. The hollow cylinder 14 has its outer circumferential surface covered entirely or partly with an outer tube 16, which is optically transparent to the laser beam. A cap 30 is sealingly fixed to the distal end of the hollow cylinder 14. The cap 30 has an optically transparent front window 32 for observing a forward region when the insert portion 1103 is inserted into the body cavity U (e.g., urethra).

The insert portion 1103 houses therein a pair of walls 40, 41 spaced laterally from each other, defining an inner space therebetween in the insert portion 1103. The insert portion 1103 also houses therein a laser beam irradiation portion 20 with a reflecting surface 21, a reciprocatingly movable member 23 which supports the laser beam irradiation portion 20 thereon, a monorail pipe 25 along which the reciprocatingly movable member 23 is reciprocatingly movable in the longitudinal direction of the insert portion 1103, nonparallel grooves 42 for changing the angle of the laser beam irradiation portion 20 so that the laser beam reflected by the laser beam irradiation portion 20 is applied to the target region at all times, an endoscope 6 for observing the living tissue, and coolant lumens. The laser beam irradiation portion 20 is rotatably supported on a pair of pivots 27 fixed to respective left and right sides of the reciprocatingly movable member 23 that is fixed to the distal end of the optical fiber 12 (energy emitter). The laser beam irradiation portion 20 has a pair of lugs 26 mounted on respective left and right sides thereof and slidably fitted respectively in the nonparallel grooves 42 defined in the walls 40, 41. The nonparallel grooves 42 extend out of parallel with the longitudinal axis of the insert portion 103.

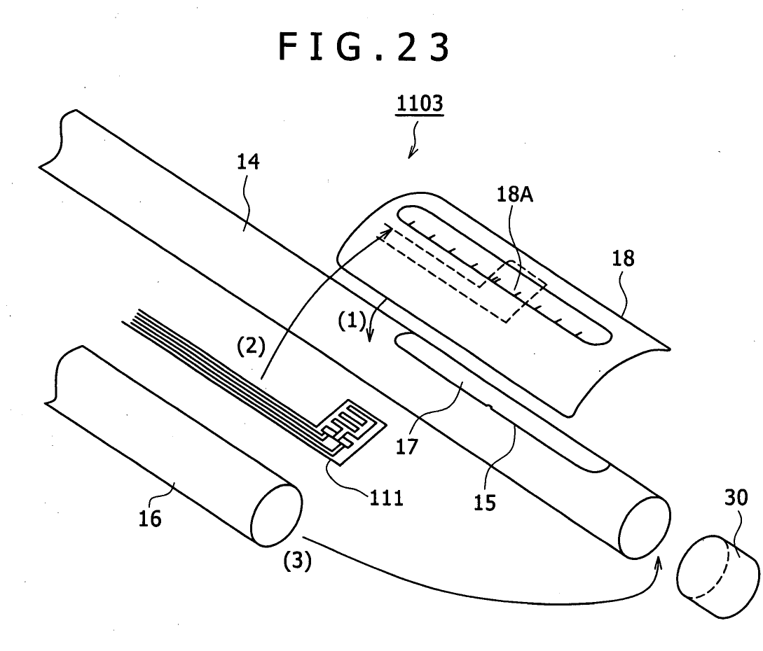

Of the major components described above, structural details of the laser beam irradiating window 17 and the temperature sensor (aluminum sensor) 111, features thereof, and processes of manufacturing them will be described below. The description of other components disposed in the insert portion 1103, i.e., the reflecting surface 21 of the laser beam irradiation portion 20, the monorail pipe 25, the reciprocatingly movable member 23, and the nonparallel grooves 42, and the description of the relationship between the movement of the reflecting surface 21 of the laser beam irradiation portion 20 and the direction in which the laser beam is applied, are the same as those in the first embodiment, and will be omitted below.

[Laser Beam Irradiating Window (FIG. 23)]

First, a process of applying the temperature sensor (aluminum sensor) 111 to the laser beam irradiating window 17 will be described below. FIG. 23 is illustrative of a process of forming the laser beam irradiating window 17 by applying the window seal with scale 18 to the opening portion 15 of the hollow cylinder 14 and then placing the temperature sensor (aluminum sensor) 111 in a given position within the window above the window seal with scale 18.

The window seal with scale 18 whose reverse side is coated with an adhesive is bonded to an area including the opening portion 15 of the hollow cylinder 14 and fixed thereto, as indicated at (1) in FIG. 23. The window seal with scale 18 should preferably be made of a synthetic resin film with a smooth surface, e.g., a film of polyester, polycarbonate, polyethylene terephthalate (PET), or the like, which is clear, colorless, and optically transparent to a laser beam. Particularly, a PET film is preferable as the material of the window seal with scale 18. The adhesive used may be any of various adhesives insofar as they can firmly bond the window seal with scale 18 to the hollow cylinder 14 to prevent the coolant circulating in the hollow cylinder 14 from leaking out of the laser beam irradiating window 17.

Then, as indicated at (2) in FIG. 23, the temperature sensor (aluminum sensor) 111 is bonded to the window seal with scale 18 at a position (indicated by the dotted lines in FIG. 23) corresponding to the window thereof, using the adhesive referred to above. Finally, the outer tube 16 is placed over the hollow cylinder 14, as indicated at (3) in FIG. 23, after which the outer tube 16 is thermally shrunk to press the temperature sensor (aluminum sensor) 111 in position. The cap 30 is put on the hollow cylinder 14. In this manner, the temperature sensor (aluminum sensor) 111 is fixed to the window seal with scale 18 at a position (indicated by the dotted lines in FIG. 23) in the window.

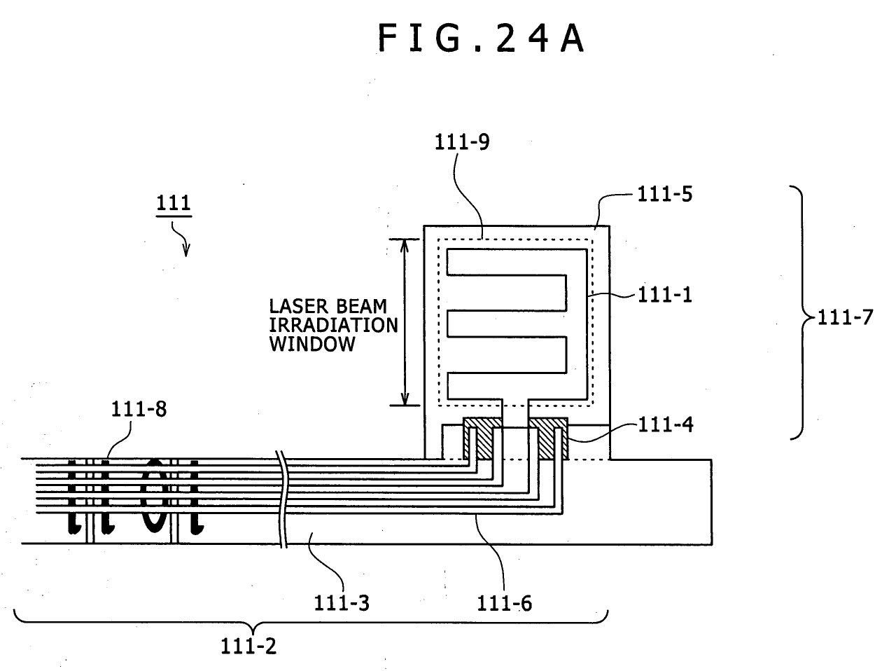

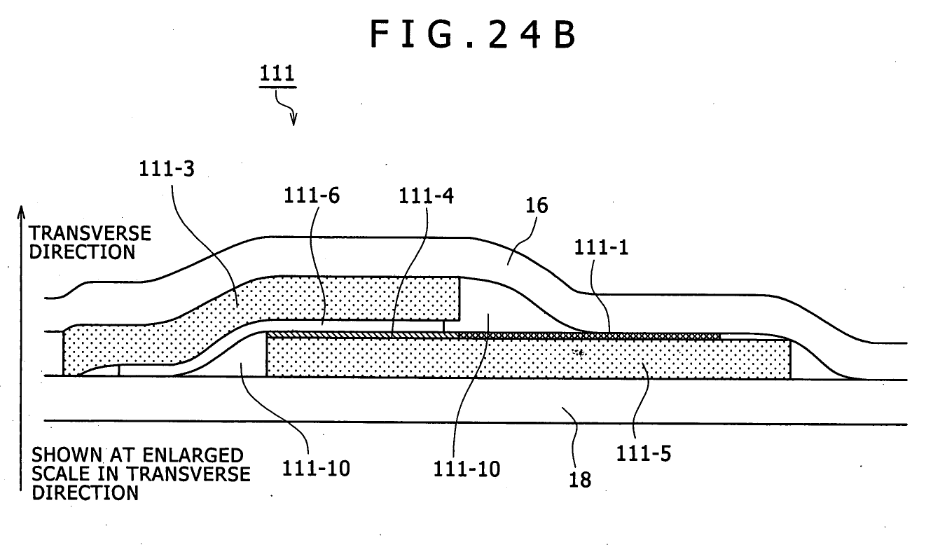

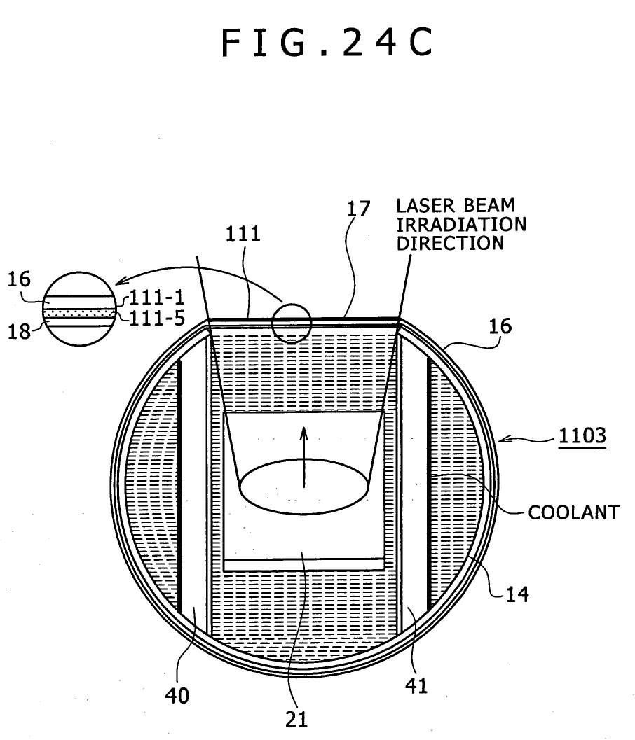

Instead of the window seal with scale 18 described above, the graduated glass strip 19A or 19B as shown in FIG. 5 may be set in the opening portion 15, and then the temperature sensor (aluminum sensor) 111 may be bonded by an adhesive to the graduated glass strip 19A or 19B at a position (indicated by the dotted lines in FIG. 23) corresponding to the window. Finally, the outer tube 16 may be placed over the hollow cylinder 14, after which the outer tube 16 may be thermally shrunk to press the temperature sensor (aluminum sensor) 111 in position.

[Structure of Temperature sensor (Aluminum Sensor) (FIGS. 24A through 24C)]

Structural details of the temperature sensor (aluminum sensor) will be described below. FIG. 24A is a front elevational view of the temperature sensor (aluminum sensor) 111. FIG. 24B shows structural details in a transverse direction of the temperature sensor (aluminum sensor) 111 that is sandwiched between the window seal with scale 18 and the outer tube 16. For illustrative purposes, the temperature sensor (aluminum sensor) 111 is shown at an enlarged scale in the transverse direction thereof in FIG. 24B. FIG. 24C shows the temperature sensor (aluminum sensor) 111 attached to the outer surface of the laser beam irradiating window 17 of the hollow cylinder 14, and fixed in position by the outer tube 16.

As shown in FIG. 24A, the temperature sensor (aluminum sensor) 111 is constructed of the conductor assembly 111-2 and a temperature measuring unit 111-7. The conductor assembly 111-2 and the temperature measuring unit 111-7 are electrically connected to each other by joining electrodes 111-4 and conductors 111-6 to each other. The electrodes 111-4 and the conductors 111-6 may be joined to each other by an anisotropic conductive material including a thermosetting epoxy resin with conductive particles dispersed therein (generally known as ACP or ACF resin). It is especially preferable if the metal is aluminum because it can't be soldered. FIG. 24A shows an example of the configurations of the temperature measuring element 111-1 and the electrodes 111-4, and these configurations may be designed freely depending on the region to be measured.

The temperature measuring unit 111-7 is constructed of a thin-film substrate 111-5, the temperature measuring element 111-1, and the electrodes 1114. The temperature measuring element 111-1 and the electrodes 111-4 may be electrically connected to each other by being integrally manufactured or by being separately manufactured and then joined to each other. For integrally manufacturing the temperature measuring element 111-1 and the electrodes 111-4, an aluminum film is evaporated on the thin-film substrate 111-5, patterned to a predetermined shape, and then etched. The thin-film substrate 111-5 should preferably be made of a synthetic resin film with a smooth surface, e.g., a film of polyester, polycarbonate, polyethylene terephthalate (PET), polyethylene (PE), polypropylene (PP), polyamides or the like. It should preferably excel in optical penetration, thermal conduction and heatproof ability. Especially, the difference of thermal expansion between the radical material and the metal for the temperature sensor should be small to prevent the metal from flaking off from the radical material. Particularly, a PET film is preferable as the material of the thin-film substrate 111-5. The thickness of the thin-film substrate 111-5 is in the range from 16 to 80 μm, or preferably in the range from 38 to 50 μm. The temperature measuring element 111-1 preferably has a line width in the range from 5 to 40 μm and a total length in the range from 50 to 100 mm. Preferably, the temperature measuring element 111-1 has a resistance in the range from 100 to 1000 Ω. The temperature measuring unit 111-7 has a temperature measuring region 111-9 to be measured by the temperature measuring element 111-1, and the temperature measuring region 111-9 has an area of 9 mm², for example. The area of the temperature measuring region should preferably be greater than the width of the laser beam that passes through the laser beam irradiating window or should preferably be greater than the diameter of the laser beam spot and smaller than the width of the laser beam irradiating window. If the temperature measuring element 111-1 (having a line width of 20 μm and a total length of 85 mm) shown in FIG. 24A is disposed to cover a wide region (the area of the temperature measuring region: 9 mm²) extending from a position near the upper end of the laser beam irradiating window 17 to a position near the lower end of the laser beam irradiating window 17, as shown in FIG. 22, then since any portion of the laser beam blocked by the temperature measuring element 111-1 is very small, the irradiation of the living tissue with the laser beam is not substantially inhibited. For example, when a laser beam of 25 W is applied, the portion of the laser beam blocked by the temperature measuring element 111-1 is represented by about 0.15% (34 mW) of the irradiation window.

The conductor assembly 111-2 is constructed of the thin-film substrate 111-3 made of an insulating material such as polyimide, nylon, polyethylene, PET, or the like, and four conductors 111-6 mounted on the thin-film substrate 111-3 and each in the form of a strip of a conductive material. Two of the four conductors 111-6 are used to detect a voltage, and the other two are used to introduce a constant current. The four conductors 111-6 may be replaced with two conductors 111-6. The thin-film substrate 111-2 has a plurality of position (depth) markers 111-8 thereon for the user to easily read the length of the temperature sensor (aluminum sensor) 111 which has been inserted into a living body, as shown in FIG. 24A. The thin-film substrate 111-3 includes a thin film having a thickness in the range from 10 to 40 μm, preferably from 15 to 25 μm, and can flexibly be bent.

[Features of Temperature Sensor (Aluminum Sensor) (Thickness)]

Features of the temperature sensor (aluminum sensor) 111 will be described below. According to a first feature of the temperature sensor (aluminum sensor) 111, the thickness of the temperature sensor can be reduced. A thin-film metal resistor that can be used as the temperature measuring element is a thin metal film of Al, Pt, Ti, W, Ni, Co, Cu, Ag, Au, or the like, for example. Since the thin-film metal resistor itself has a very small thickness in the range from 0.2 to 3 μm, the temperature sensor can be reduced in thickness. As metal has a large laser beam reflectance (e.g., 90% for Al), a metal film used as the temperature measuring element does not need to be covered with a laser beam shield plate, so that the temperature sensor can further be reduced in thickness. Additionally, it should preferably be high in light reflectance rate, electric resistance, and resistance temperature coefficient (TCR). As for this metal, the melting point should be low and the thermal conductivity should be high.

An example of thicknesses of the components of the temperature sensor (aluminum sensor) 111 wherein the thin-film metal resistor is made of aluminum will be described below. The thin-film substrate 111-5 has a thickness in the range from 16 to 80 μm, or preferably in the range from 38 to 50 μm, each of the temperature measuring element 111-1 and the electrodes 1114 has a thickness in the range from 0.2 to 3 μm, or preferably in the range from 0.5 to 1.5 μm. The conductors 111-6 have a thickness in the range from 10 to 20 μm. The thin-film substrate 111-3 has a thickness in the range from 10 to 20 μm. As shown in FIG. 24B, the thickness of the temperature measuring unit 111-7 (the temperature measuring element 111-1+the thin-film substrate 111-3) of the temperature sensor (aluminum sensor) 111 can be reduced to a value in the range from 16 to 83 μm.

FIG. 24C shows the temperature sensor (aluminum sensor) 111 mounted on the outer surface of the laser beam irradiation window 17 of the hollow cylinder 14, and secured in place by the outer tube 16. In FIG. 24C, the hollow cylinder 14 has an outside diameter of 7 mm, the temperature sensor 111 has a thickness of 20 μm, and the outer tube 16 has a thickness of 20 μm. As can be seen from FIG. 24C, even with the temperature sensor (aluminum sensor) 111 mounted on the outer surface of the laser beam irradiation window 17, the diameter of the insert portion 1103 is substantially the same as the outside diameter of the hollow cylinder 14. Therefore, when the insert portion 1103 with the temperature sensor 111 installed thereon is inserted into a living body, the possibility that the surface of the living body will be damaged by the temperature sensor 111 is reduced to the possibility that it will be damaged by the insert portion 1103 which is free of the temperature sensor 111. Since the temperature sensor 111 is fixed in position by the outer tube 16, the temperature sensor 111 is prevented from being positionally displaced when it is in use.

In FIG. 24C, the laser beam irradiating window 17 is shown as being flat. However, even if the laser beam irradiating window 17 is of an arcuately curved cross section matching the circular cross section of the hollow cylinder 14 and the temperature sensor 111 is mounted on the outer surface of the laser beam irradiating window 17, because the thickness of the temperature sensor 111 is reduced to about 20 μm, the possibility that the surface of the living body will be damaged by the temperature sensor 111 is reduced to the possibility that it will be damaged by the insert portion 1103 which is free of the temperature sensor 111.

[Second Feature of Temperature Sensor (Aluminum Sensor) (Measuring Region)]

A second feature of the temperature sensor (aluminum sensor) 111 will be described below. According to the second feature of the temperature sensor (aluminum sensor) 111, since a long thin-film metal resistor having a small line width is employed, it is possible to measure the temperature of a wide region (surface region or preferably a region wider than the diameter of the laser beam spot and smaller than the width of the laser beam irradiation window). For example, if the resistance of the thin-film metal resistor is in the range from 100 to 1000 Ω and the thin-film metal resistor is made of aluminum, then the thin-film metal resistor can be designed to have a line width ranging from 5 to 40 μm and a length in the range from 50 to 100 mm. For example, the thin-film resistor of aluminum having a shape shown in FIG. 24A (the line width of 20 μm and the length of 85 mm) is capable of measuring the temperature of a region (surface region) having a size of 3×3 mm.

The thin-film metal resistor made of aluminum is installed in a wide area (the area of the temperature measuring region: 9 mm²) extending from a position near the upper end of the laser beam irradiating window 17 to a position near the lower end of the laser beam irradiating window 17, as shown in FIG. 22. The temperatures sensor 111 thus constructed is capable of directly measuring the maximum temperature of the surface of the living tissue that is held in contact with the laser beam irradiation window 17.

When the temperature of a wide area (surface area) is measured by the temperature sensor 111 wherein the temperature measuring region 111-9 shaped as shown in FIG. 24A is disposed on the laser beam irradiation window 17, any portion of the laser beam that is obstructed by the temperature measuring element 111-1 is reduced because the line width of the thin-film metal resistor made of aluminum is small, i.e., 20 μm. For example, when a laser beam of 25 W is applied, the portion of the laser beam which is blocked by the temperature measuring element 111-1 shaped as shown in FIG. 24A is represented by about 0.15% (34 mW) of the irradiation window. Most of the laser beam is thus not obstructed by the thin-film metal resistor made of aluminum, but is applied to an irradiation target position.

If the temperature sensor (aluminum sensor) 111 capable of measuring the temperature of a wide region (surface region) is employed, then variations of temperature measurement due to manufacturing variations can be reduced. Manufacturing variations include variations of tilt of the laser beam irradiation portion 20 with respect to the laser beam irradiation window 17, and variations of the thickness of the temperature measuring unit. If such manufacturing variations occur, then when the temperature sensor measures the temperature of a small region (spot), the measured temperature is affected by the manufacturing variations. If insert portions are changed and used as when a plurality of insert portions are replaced and used, since the measured temperature is affected by the manufacturing variations, the temperature cannot accurately be measured. However, since the temperature sensor (aluminum sensor) 111 includes a long thin-film metal resistor having a small line width, it can measure the temperature of a wide region (surface region), and hence can measure, accurately at all times, the maximum temperature of the surface of the living tissue that is held in contact with the laser beam irradiation window 17 even though manufacturing variations occur.

[Process of Manufacturing Aluminum Sensor]

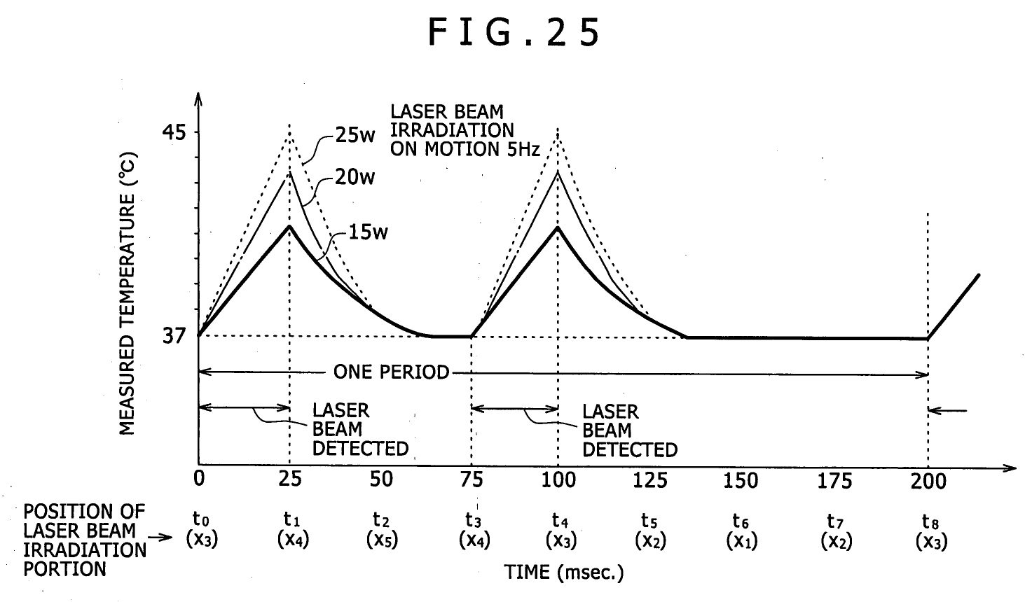

A process of manufacturing the temperature measuring unit of the temperature sensor (aluminum sensor) 111 will be described below. For simultaneously manufacturing the temperature measuring unit 111-7 and the electrodes 111-4 of the temperature sensor (aluminum sensor) 111 shaped as shown in FIG. 24A, an aluminum layer is formed by vacuum evaporation on a thin-film substrate made of an optically transparent resin such as PET or the like and having a thickness in the range from 16 to 80 μm, or preferably in the range from 38 to 50 μm. The aluminum layer has a thickness in the range from 0.2 to 3 μm, or preferably in the range from 0.5 to 1.5 μm. Then, the aluminum layer is coated with a resist, after which the thin-film substrate is exposed to light by photolithography to form a pattern on the resist. The exposed resist is then etched away, and, using the remaining resist as a mask, the aluminum layer below the mask is etched by wet or dry etching, after which the unwanted resist is removed. In this manner, the temperature measuring unit 111-7 and the electrodes 111-4 of the temperature sensor (aluminum sensor) 111 shaped as shown in FIG. 24A are produced. The produced temperature measuring element 111-1 has a resistance in the range from 100 to 1000 Ω, a line width in the range from 5 to 40 μm, and a total length in the range from 50 to 100 mm. The temperature measuring surface region 111-9 has a size of 3×3 mm. The shapes of the temperature measuring element 111-1 and the electrodes 111-4 may be designed freely depending on the region to be measured.

[Measurement of Temperature of Surface Layer Irradiated with Laser Beam (FIG. 25)]

An example of results produced when temperatures were measured using the temperature sensor (aluminum sensor) 111 is shown in FIG. 25. FIG. 25 illustrates by way of example time-dependent changes of temperatures measured by the temperature sensor (aluminum sensor) 111 when the temperature sensor (aluminum sensor) 111 was mounted on the laser beam irradiation window 17 (having a length of 30 mm in the longitudinal direction) and the laser beam irradiation window 17 was reciprocally moved (see FIG. 2) in the longitudinal direction of the insert portion 1103 at a frequency of 5 Hz (200 msec.) (see FIG. 22 for the position of the laser beam irradiation portion shown in FIG. 25). The temperature measuring region 111-9 (see FIG. 24A) of the temperature sensor (aluminum sensor) 111 has a size of 3×3 mm, and the output of the laser beam is in the range from 15 W to 25 W. Since the laser beam irradiation window 17 reciprocally moves in the longitudinal direction of the insert portion 1103 in FIG. 22, the temperature sensor (aluminum sensor) 111 intermittently detects the laser beam in a time period from t0 to t1 and a time period from t3 to t4. During the detecting periods (irradiation periods), the temperature sensor aluminum sensor) 111 (laser beam reflectance: 90%) absorbs part of the laser beam and is heated, so that the temperature increases. During time periods from t2 to t3 and from t4 to t8, since the temperature sensor (aluminum sensor) 111 does not detect the laser beam, the temperature measured by the temperature sensor (aluminum sensor) 111 drops to the ambient temperature. In the example shown in FIG. 25, the temperature measured by the temperature sensor (aluminum sensor) 111 is substantially equal to the temperature prior to the laser beam irradiation at a time t3 or a time t6 (the temperature of the surface of the living body held in contact with the temperature sensor). Therefore, when the temperature T measured in a time period from t6 to t8 is measured as the temperature (ambient temperature) of the surface of the living body held in contact with the temperature sensor while the living body is being irradiated with the laser beam, changes in the surface layer temperature while the living body is being irradiated with the laser beam can accurately be measured without being affected by the heating of the temperature sensor (aluminum sensor) 111 irradiated with the laser beam.

In FIG. 25, the measured temperature representing the temperature (ambient temperature) of the surface of the living body held in contact with the temperature sensor before the living body is irradiated with the laser beam increases when irradiated with the laser beam, and a time that is consumed until the increased temperature drops to a temperature equal to the temperature (ambient temperature) of the surface of the living body when the laser beam irradiation stops is defined as a response speed. The response speed of the temperature sensor (aluminum sensor) 111 having the structure shown in FIG. 24A is about 50 msec.

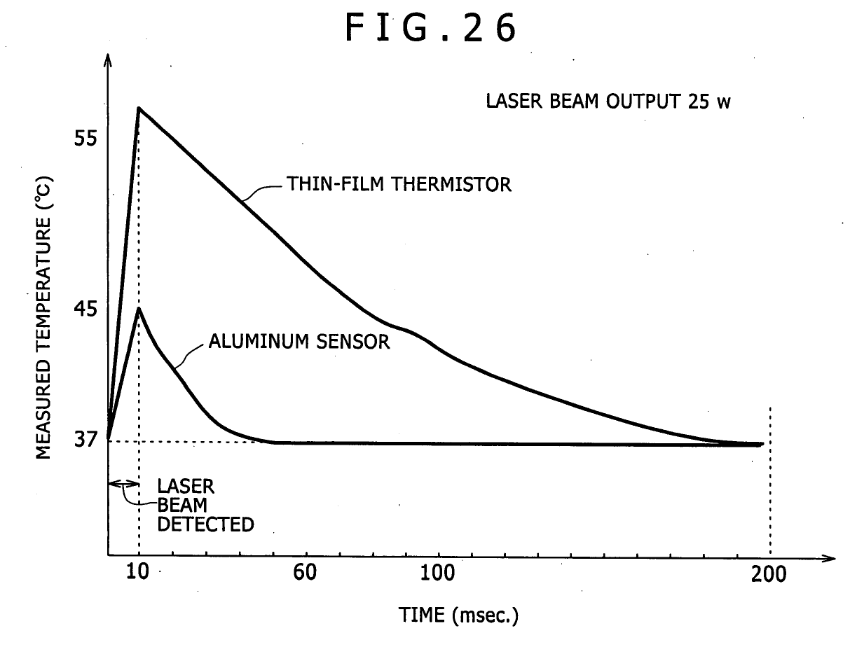

FIG. 26 is a diagram showing for comparison response speeds of the temperature sensor 11 (thin thermistor) used in the first embodiment and the temperature sensor (aluminum sensor) 111 according to the present embodiment. It can be seen from FIG. 26 that the response speed of the temperature sensor (aluminum sensor) 111 is faster than the response speed of the thin thermistor. Therefore, the temperature sensor (aluminum sensor) 111 can be used as a sensor suitable for measuring the surface layer temperature when the laser beam irradiation portion 20 reciprocally moves at a high speed. When temperature sensor 11 (thin thermistor) is used, the slower response speed thereof can be made up for by corrective calculations. Specifically, an equation for estimating the surface layer temperature, e.g., the equation (1), may be used to calculate the maximum temperature from the measured temperature. Accordingly, either the temperature sensor 11 (thin thermistor) or the temperature sensor (aluminum sensor) 111 can be used as a sensor suitable for measuring the surface layer temperature when the laser beam irradiation portion 20 reciprocally moves.

[Temperature Control System (FIG. 11)]

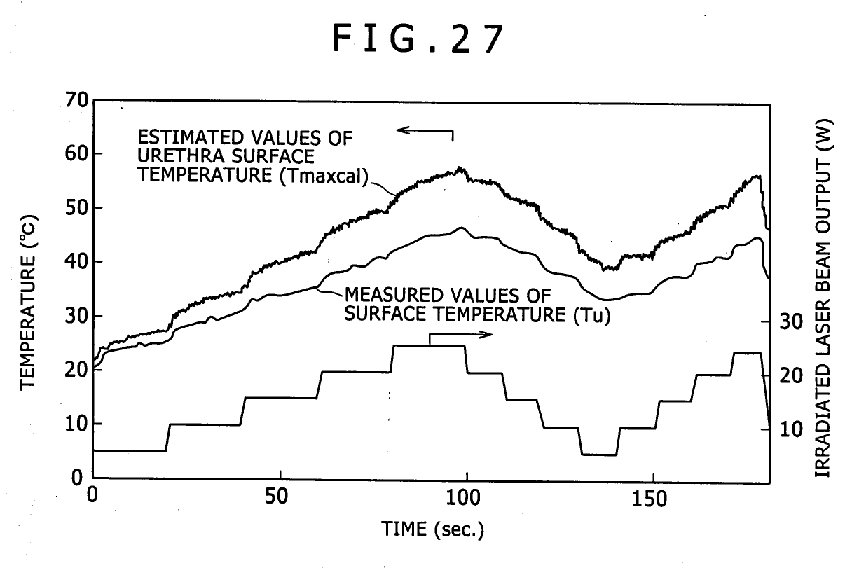

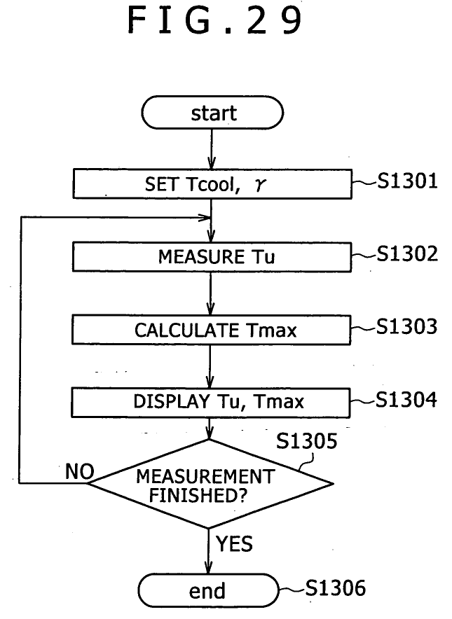

A process of estimating a surface temperature and various control processes with the energy irradiating medical apparatus 110 using the measured temperature will be described below. Since a control circuit of the energy irradiating medical apparatus 110 is the same as the control circuit of the energy irradiating medical apparatus 10 according to the first embodiment described above with reference to FIG. 11, the control circuit of the energy irradiating medical apparatus 110 will not be described below. [Estimation of urethra surface temperature (FIGS. 27 through 29)]

A process of estimating a urethra surface temperature from the surface temperature actually measured by the temperature sensor (aluminum sensor) 111 mounted on the insert portion 1103 when the doctor treats the urethra with heat using the energy irradiating medical apparatus 110 will be described below.

Since the temperature sensor (aluminum sensor) 111 is disposed to cover a region (preferably a region wider than the diameter of the laser beam spot and narrower than the width of the laser beam irradiation window) shown in FIG. 22 on the laser beam irradiation window 17, the surface temperature Tu measured when the laser beam is applied is considered to directly represent the surface temperature of the living tissue (the surface temperature of the urethra). Actually, however, since the temperature sensor is sandwiched between the window seal with scale 18 and the outer tube 16, as shown in FIG. 24B, an actual urethra surface temperature Tmax is higher than the measured surface temperature Tu. Therefore, it is necessary to estimate an actual urethra surface temperature Tmax from the measured surface temperature Tu. A process of estimating an actual urethra surface temperature from the measured surface temperature Tu will be described below with reference to FIGS. 27 through 29.

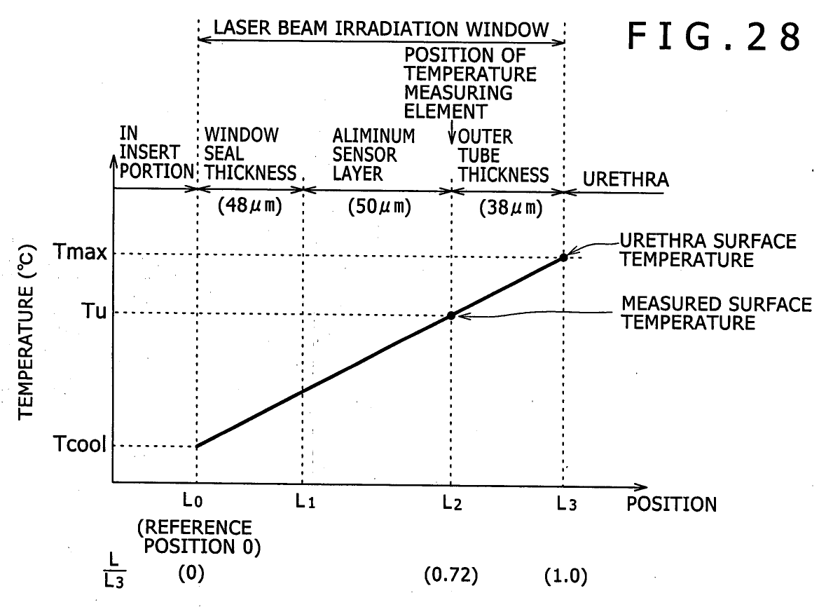

FIG. 28 shows the positional relationship between the temperature measuring element 111-1 of the temperature sensor (aluminum sensor) 111 and the urethra surface. In FIG. 28, the horizontal axis represents the positions of the laser beam irradiation window and the urethra surface, and the vertical axis represents the temperature. To produce results shown in FIG. 28, the window seal with scale 18 had a thickness of 48 μm, the temperature sensor (aluminum sensor) had a thickness of 50 μm (the thin-film substrate 111-5: 49 μm, the temperature measuring element 111-1: 1 μm), and the outer tube 16 had a thickness of 38 μm. Therefore, the temperature measured by the temperature measuring element 111-1 is not the temperature of the urethra surface, but the temperature at a position that is spaced 38 μm from the urethra surface (the temperature within the insert portion).

If the inner surface (L0 in FIG. 28) of the insert portion is represented as a reference position, the position of the temperature measuring element 111-1 as L2, and the position of the urethra surface as L3, then the ratio of the length (L2) from the inner surface of the insert portion to the temperature measuring element 111-1 to the length (L3) from the inner surface of the insert portion to the urethra surface is defined as □ (corrective coefficient), which is given as follows:
γ=L2/L3=0.72 (3)
Using the corrective coefficient γ according to the equation (3), the urethra surface temperature Tmax can be obtained from the measured surface temperature Tu by the following equation:
Tmax=Tcool+(Tu−Tcool)/γ (4)
where Tcool represents the temperature of the coolant for cooling the interior of the insert portion 1103, and is 20° C., for example.

FIG. 27 shows values plotted at desired times as the surface temperature Tu measured when the living tissue is irradiated with the laser beam and values plotted as the estimated urethra surface temperature Tmaxcal that is calculated from the measured surface temperature Tu according to the equation (4). In the example shown in FIG. 27, the output of the laser beam increased stepwise from 0 to 25 W, and thereafter decreased stepwise from 25 to 0 W. Changes in the measured surface temperature Tu indicate that the surface temperature Tu was accurately measured depending on the output of the laser beam. It can thus be understood that the maximum temperature Tmax of the urethra surface can accurately be estimated according to the equation (4) from the surface temperature Tu that is measured at desired times when the living tissue is irradiated with the laser beam.

Based on the above experimental results, a control program for calculating the maximum temperature Tmax of the urethra surface from the surface temperature Tu measured when the living tissue is irradiated with the laser beam was generated and stored in the ROM 202. FIG. 30 shows a processing sequence that is carried out by the CPU 201 according to the control program. The processing sequence is started when an execution command or an initial value for executing the control program is entered from the control console when the doctor treats the living tissue with heat using the energy irradiating medical apparatus.

In step S1301, Tcool (e.g., 20° C.) and the corrective coefficient β (e.g., 0.72) are set. In step S1302, the surface temperature Tu is measured. In step S1303, the urethra surface temperature Tmax is calculated according to the equation (4). In step S1304, the measured surface temperature Tu and the calculated urethra surface temperature Tmax are displayed on the display. If a next measuring cycle is to be performed in step S1305, then control goes back to step S1302 to repeat the processing from step S1302. If the measuring process is to be finished in step S1305, then control goes to step S1306 where the processing sequence is put to an end.

[Estimation of Temperature of Deep Region of Living Body]

The energy irradiating medical apparatus 110 according to the present embodiment is capable of monitoring irradiation timing for applying the laser beam from the value of the surface temperature measured by the temperature sensor (aluminum sensor) 111 mounted on the insert portion 1103 when the doctor treats the urethra with heat, as described above with reference to FIGS. 17 and 18 with regard to the energy irradiating medical apparatus 10 according to the first embodiment, and determining whether the irradiation timing is correct or not. However, the monitoring process is the same as the process described above with reference to FIGS. 17 and 18, and will not be described below.

[Controlling of Laser Beam Output Value (FIGS. 19 and 20)]

The energy irradiating medical apparatus 110 according to the present embodiment is also capable of controlling the output value of the laser beam when the living body is irradiated with the laser beam (e.g., to heat the living tissue according to the temperature increasing pattern shown in FIG. 19), from the surface temperature measured by the temperature sensor (aluminum sensor) 111 mounted on the insert portion 1103 when the doctor treats the urethra with heat, as described above with reference to FIGS. 19 and 20 with regard to the energy irradiating medical apparatus 10 according to the first embodiment. However, the controlling process is the same as the process described above with reference to FIGS. 19 and 20, and will not be described below.

The embodiments described above are not described in order to limit the present invention, but various modifications may be made therein within the technical concept of the present invention. The energy irradiating medical apparatus according to the present invention should preferably be applied to the treatment of a prostate gland with heat while reducing heat-induced damage to a normal tissue such as a urethra or a rectum that is present in the vicinity of the prostate gland, in the treatment of a prostatic disease such as benign prostatic hypertrophy or prostatic cancer.

[Summary of Temperature Sensor According to Second Embodiment]

The features of the temperature sensor (thin-film metal resistor, metal sensor) used in the energy irradiating medical apparatus 110 according to the second embodiment will be summarized as follows: The energy irradiating medical apparatus 110 is capable of directly accurately measuring the surface temperature of a living tissue while it is being treated with heat, for an increased therapeutic effect, when the insert portion having the laser beam irradiation window for applying the laser beam is inserted from a lumen such as a urethra, and the laser beam is applied from the laser beam irradiation window to the living tissue to treat benign prostatic hypertrophy with heat. The temperature measuring unit of the metal sensor employs a thin-film metal resistor as a temperature measuring element, and has a thickness ranging from 0.2 to 3 μm which is suitable for making the temperature sensor smaller in size. The temperature measuring unit is suitable for measuring a wide region (surface region) having an area of 9 mm², for example. The temperature measuring element of the thin-film metal resistor may be of a simple structure as there is no need for a laser beam shield plate because it has a large laser beam reflectance (90% for Al). As shown in FIG. 24A, since the thin-film metal resistor is of a slender configuration having a line width in the range from 5 to 40 μm and a total length in the range from 50 to 100 mm, it does not obstruct the irradiation of the laser beam even if installed in a wide region (e.g., 3 mm×3 mm) over the laser beam irradiation window, and does not obstruct the heat treatment. For example, an energy loss when a laser beam of 25 W is applied is 34 mW (0.15%). The temperature measuring unit has a high response speed of 50 msec., and is suitable for the measurement of the maximum temperature of the surface of a lumen by intermittently detecting the laser beam that is applied from the laser beam irradiation portion which reciprocatingly moves at a frequency ranging from 3 to 10 Hz, when the living tissue is irradiated with the laser beam.

The maximum temperature of the surface of the lumen and the temperature (laser beam irradiation target temperature) of the deep region heated by being irradiated with the laser beam can be estimated from the measured temperature of the surface of the lumen. Therefore, the energy irradiating medical apparatus can continuously estimate and display, on a display, time-dependent changes of the maximum temperature of the surface of the lumen and the temperature of the deep region. The energy irradiating medical apparatus can be controlled so that when the measured temperature exceeds a preset temperature, the energy irradiating medical apparatus issues a light or sound warning to prompt the operator to pay attention or stops applying the laser beam. Therefore, the living tissue is prevented from being irreversibly damaged due to denaturization of protein (the living tissue is irreversibly damaged if exposed to the temperature of 55° C. for about 20 seconds, the temperature of 50° C. for about 5 minutes, and the temperature of 48° C. for about 10 minutes). The doctor can change laser beam irradiating conditions for the treatment with heat so as not to damage the urethra, by monitoring the maximum temperature of the surface of the lumen that is displayed on the display. The doctor can also monitor the effectiveness of the treatment with heat or control the application of the laser beam depending on the temperature of the deep region by monitoring the temperature of the deep region displayed on the display. For example, if the temperature of the deep region is too low, the doctor can intensify the application of the laser beam, and if the temperature of the deep region has reached a target temperature, the doctor can stop applying the laser beam.

Inasmuch as the temperature measuring unit of the metal sensor is suitable for the measurement of a wide region (surface region), even if the insert portion suffers manufacturing variations when it is manufactured, the temperature of the temperature measuring unit is not changed due to such manufacturing variations.

Though the energy irradiating medical apparatus according to the present embodiment is of a structure that is simple and inexpensive to manufacture, it is capable of safely treating a living tissue with heat by accurately measuring the temperature of the living tissue while it is being treated with heat. Since the temperature measuring unit is thin, the insert portion may be reduced in size to reduce the pain which the patient suffers when the insert portion is inserted into the patient. As the temperature measuring element does not need to be connected to two leads and placed in a tangle-free manner in a protective tube unlike the conventional structure, the insert portion can be reduced in size. Since the temperature measuring element is not disposed in the insert portion, it is less affected by the coolant and can measure the temperature of the surface of the living body with high accuracy. Since the temperature sensor is not required to directly thrust into a living tissue to measure the temperature of the living tissue, the living tissue is prevented from being damaged by thrusting thereinto and also from a side effect due to an infectious disease.

While preferred embodiments of the invention have been described using specific terms, such description is for illustrative purposes only, and it is to be understood that changes and variations may be made, and equivalents employed, without departing from the spirit and scope of the invention as recited in the following claims.

1. An energy irradiating medical equipment comprising an insert portion to be inserted into a living body, an energy emitter adapted to be connected to an energy generator to emit energy to irradiate living tissue of the living body, and a temperature sensor disposed on said insert portion,
said temperature sensor comprising:
a flexible thin-film substrate;
at least first and second conductors disposed on said thin-film substrate; and
a temperature measuring unit electrically coupled to said at least first and second conductors.

2. The energy irradiating medical equipment according to claim 1, wherein said insert portion comprises an energy irradiation window through which the energy emitted from the energy emitter is directed to the living tissue, said temperature measuring unit being disposed in a peripheral region within said energy irradiation window.

3. The energy irradiating medical equipment according to claim 2, wherein said temperature measuring unit comprises:
first and second electrodes bonded and electrically coupled respectively to at least the first and second conductors disposed on said thin-film substrate; and
a substantially plate-shaped thermistor element made of a metal oxide;
said first and second electrodes being electrically coupled to said thermistor element.

4. The energy irradiating medical equipment according to claim 2, wherein said thermistor element possesses a first surface disposed on said first electrode, said first electrode is bonded and electrically coupled to said thermistor element, said thermistor element possesses a second surface opposite to said first surface, with said second electrode being disposed on said second surface, and said second electrode is not bonded to, but electrically coupled to said thermistor element.

5. The energy irradiating medical equipment according to claim 2, wherein said flexible thin-film substrate is bent to place said second electrode on a second surface of said thermistor element which is opposite to a first surface.

6. The energy irradiating medical equipment according to claim 1, wherein said insert portion includes an energy irradiation window through which the energy emitted from the energy emitter is directed to the living tissue, said thin-film substrate being disposed outwardly of said energy irradiation window and along a longitudinal direction of said insert portion.

7. The energy irradiating medical equipment according to claim 4, further comprising an output tube thermally shrunk around and covering said insert portion to press said thermistor element and said second electrode against each other to electrically couple said thermistor element and said second electrode to each other.

8. The energy irradiating medical equipment according to claim 3, further comprising a thin metal film for shielding said thermistor element from said energy.

9. The energy irradiating medical equipment according to claim 8, wherein said thin metal film is disposed on said thin-film substrate, and said thin-film substrate is bent to cover said thermistor element with said thin metal film.

10. The energy irradiating medical equipment according to claim 1, wherein said insert portion comprises a hollow cylinder and an opening portion defined in a side wall of said hollow cylinder forming an energy irradiation window through which the energy emitted from the energy emitter is directed to the living tissue.

11. The energy irradiating medical equipment according to claim 10, wherein an optically transparent resin film is applied to said hollow cylinder in covering relation to said opening portion.

12. The energy irradiating medical equipment according to claim 11, wherein said resin film is provided with a scale.

13. The energy irradiating medical equipment according to claim 11, further comprising an outer tube covering said resin film.

14. The energy irradiating medical equipment according to claim 1, wherein said thin-film substrate comprises depth markers for indicating the length by which the insert portion is inserted into the living body by the user.

15. The energy irradiating medical equipment according to claim 1, wherein said insert portion includes an energy irradiation window through which the energy emitted from the energy emitter is directed to the living tissue, said temperature measuring unit being disposed in a peripheral region within said energy irradiation window and comprising a plurality of temperature sensors disposed in different positions on said insert portion.

16. The energy irradiating medical equipment according to claim 1, wherein said energy emitter is an optical fiber adapted to be connected to a laser beam generator.

17. The energy irradiating medical equipment according to claim 1, wherein said temperature measuring unit comprises a thin-film metal resistor.

18. An energy irradiating medical equipment comprising an insert portion to be inserted into a living body, an energy emitter adapted to be connected to an energy generator to emit energy to irradiate living tissue of the living body, and a temperature sensor disposed on said insert portion,
said temperature sensor comprising:
a flexible thin-film substrate;
at least first and second conductors disposed on said thin-film substrate; and
a temperature measuring unit electrically coupled to said at least first and second conductors and including a thin-film metal resistor.

19. The energy irradiating medical equipment according to claim 18, wherein said insert portion comprises an energy irradiation window through which the energy emitted from the energy emitter is directed to the living tissue, said temperature measuring unit being disposed over a region of the energy irradiation window greater than an irradiated width of the energy which passes through said energy irradiation window.

20. The energy irradiating medical equipment according to claim 18, wherein said thin-film substrate is made of an optically transparent resin which permits transmission of said energy through the thin-film substrate.

21. The energy irradiating medical equipment according to claim 18, wherein said insert portion comprises an energy irradiation window through which the energy emitted from the energy emitter is directed to the living tissue, said thin-film metal resistor covering said energy irradiation window in a range greater than a diameter of an irradiated spot of said energy, and smaller than a width of said energy irradiation window.

22. An energy irradiating medical apparatus comprising:
an insert portion to be inserted into a living body;
an energy emitter adapted to be connected to an energy generator to emit energy to irradiate living tissue of the living body;
a temperature sensor disposed on said insert portion, said temperature sensor comprising a flexible thin-film substrate, at least first and second conductors disposed on said thin-film substrate, and a temperature measuring unit electrically coupled to said at least first and second conductors; and
surface temperature estimating means for estimating a surface temperature of the living tissue which is irradiated with said energy based on a temperature measured by said temperature sensor.

23. The energy irradiating medical apparatus according to claim 22, wherein said insert portion comprises an energy irradiation window through which the energy emitted from the energy emitter is directed to the living tissue, said temperature measuring unit being disposed in a peripheral region within said energy irradiation window, said temperature measuring unit comprising a first electrode bonded to the first conductor and a second electrode electrically coupled to the first and second conductors disposed on said thin-film substrate, and a substantially plate-shaped thermistor element made of a metal oxide, said first and second electrodes being electrically coupled to said thermistor element.

24. The energy irradiating medical apparatus according to claim 22, wherein said insert portion comprises an energy irradiation window through which the energy emitted from the energy emitter is directed to the living tissue, said temperature measuring unit being disposed on said energy irradiation window, said temperature measuring unit comprising a first electrode disposed on said thin-film substrate and bonded to the first conductor, a second electrode disposed on said thin-film substrate and electrically coupled to the second conductor, and a thin-film metal resistor bonded and electrically coupled to said first and second electrodes.

25. The energy irradiating medical apparatus according to claim 22, further comprising deep region temperature estimating means for estimating a deep region temperature of a living tissue which is irradiated with said energy based on a temperature measured by said temperature sensor.

26. The energy irradiating medical apparatus according to claim 22, further comprising control means for controlling the energy applied to said living tissue based on the temperature measured by said temperature sensor.

27. The energy irradiating medical apparatus according to claim 26, wherein said energy emitter is an optical fiber which emits a laser beam, and further comprising:
irradiating means disposed in said insert portion for reflecting said laser beam with a reflecting surface and applying the laser beam through an energy irradiation window to the living tissue;
moving means for reciprocally moving said irradiating means along a longitudinal direction of said insert portion;
changing means for changing an irradiation angle of said irradiating means; and
determination means for determining whether or not the reciprocating movement of said irradiating means is correctly controlled by said moving means based on the temperature measured by said temperature sensor.

28. The energy irradiating medical apparatus according to claim 22, wherein said energy is a laser beam.

29. An energy irradiating medical equipment comprising: an insert portion possessing a size permitting the insert portion to be inserted into a living body, the insert portion comprising a hollow cylinder possessing an interior, an energy emitter positioned in the interior of the hollow cylinder and adapted to be connected to an energy generator to emit energy, a temperature sensor disposed on said insert portion, an opening provided in said hollow cylinder, and a cover covering the opening and permitting transmission therethrough of the energy emitted by the energy emitter;
said temperature sensor comprising:
a flexible thin-film substrate;
at least first and second conductors disposed on said thin-film substrate; and
a temperature measuring unit electrically coupled to said at least first and second conductors, said temperature measuring unit being positioned outside of said cover so that the cover is positioned between the interior of the insert portion and the temperature measuring unit.

30. The energy irradiating medical equipment according to claim 29, wherein said energy emitter is an optical fiber adapted to be connected to a laser beam generator forming said energy generator.

31. The energy irradiating medical equipment according to claim 29, wherein said temperature measuring unit comprises a substantially plate-shaped thermistor element made of a metal oxide.

32. The energy irradiating medical equipment according to claim 29, wherein said thin-film substrate is made of an optically transparent resin which permits transmission of said energy through the thin-film substrate.

33. An energy irradiating medical equipment comprising an insert portion possessing a size permitting the insert portion to be inserted into a living body, the insert portion comprising a hollow cylinder having an interior, an energy emitter positioned in the interior of the hollow cylinder and adapted to be connected to an energy generator to emit energy to irradiate living tissue of the living body, and a temperature sensor disposed on said insert portion,
said temperature sensor comprising:
a flexible thin-film substrate;
at least first and second conductors disposed on said thin-film substrate, said at least first and second conductors being positioned exteriorly of the hollow cylinder; and
a temperature measuring unit electrically coupled to said at least first and second conductors.

34. The energy irradiating medical equipment according to claim 33, wherein said energy emitter is an optical fiber adapted to be connected to a laser beam generator.

35. The energy irradiating medical equipment according to claim 33, wherein said temperature measuring unit comprises a substantially plate-shaped thermistor element made of a metal oxide.

36. The energy irradiating medical equipment according to claim 33, wherein said thin-film substrate is made of an optically transparent resin which permits transmission of said energy through the thin-film substrate.

37. A method for irradiating living tissue of a living body comprising:
inserting into the living body an insert portion in which a temperature sensor is disposed on the insert portion, the temperature sensor comprising a flexible thin-film substrate, at least first and second conductors disposed on the thin-film substrate and a temperature measuring unit electrically coupled to the at least first and second conductors;
emitting energy from within the insert portion and through a window in the insert portion to irradiate the living tissue with the energy; and
determining a temperature of the living tissue irradiated with energy using output from the temperature sensor.

38. The method according to claim 37, wherein the temperature measuring unit is disposed in a peripheral region within the energy irradiation window.

39. The method according to claim 37, wherein the temperature measuring element comprises a thermistor element made of a metal oxide.

40. The method according to claim 37, comprising determining a depth of insertion of the insert portion into the living body through use of depth markers on the insert portion.

41. The method according to claim 37, wherein the temperature measuring unit comprises a plurality of temperature sensors disposed in different positions on the insert portion.

42. The method according to claim 37, wherein the energy is emitted through an optical fiber located inside the insert portion.

43. The method according to claim 37, wherein the temperature measuring unit comprises a thin-film metal resistor.

44. The method according to claim 37, wherein the determination of the temperature of the living tissue in the living body comprises estimating a surface temperature of the living tissue which is irradiated with the energy based on a temperature measured by the temperature sensor.
